SEQUENCE LISTING (34 sequences)

SEQ ID NO: 1 - DNA, 1380 bp, Phytium irregulare
  CDS (1)..(1380): Delta-6-Desaturase
atggtggacctcaagcctggagtgaagcgcctggtgagctggaaggagatccgcgagcacgcgacgcccgcgaccgcgtggatcgtgattcaccacaaggtctacgacatctccaagtgggactcgcacccgggtggctccgtgatgctcacgcaggccggcgaggacgccacggacgccttcgcggtcttccacccgtcctcggcgctcaagctgctcgagcagttctacgtcggcgacgtggacgaaacctccaaggccgagatcgagggggagccggcgagcgacgaggagcgcgcgcgccgcgagcgcatcaacgagttcatcgcgtcctaccgccgtctgcgcgtcaaggtcaagggcatggggctctacgacgccagcgcgctctactacgcgtggaagctcgtgagcacgttcggcatcgcggtgctctcgatggcgatctgcttcttcttcaacagtttcgccatgtacatggtcgccggcgtgattatggggctcttctaccagcagtccggatggctggcgcacgacttcttgcacaaccaggtgtgcgagaaccgcacgctcggcaaccttatcggctgcctcgtgggcaacgcctggcagggcttcagcatgcagtggtggaagaacaagcacaacctgcaccacgcggtgccgaacctgcacagcgccaaggacgagggcttcatcggcgacccggacatcgacaccatgccgctgctggcgtggtctaaggagatggcgcgcaaggcgttcgagtcggcgcacggcccgttcttcatccgcaaccaggcgttcctatacttcccgctgctgctgctcgcgcgcctgagctggctcgcgcagtcgttcttctacgtgttcaccgagttctcgttcggcatcttcgacaaggtcgagttcgacggaccggagaaggcgggtctgatcgtgcactacatctggcagctcgcgatcccgtacttctgcaacatgagcctgtttgagggcgtggcatacttcctcatgggccaggcgtcctgcggcttgctcctggcgctggtgttcagtattggccacaacggcatgtcggtgtacgagcgcgaaaccaagccggacttctggcagctgcaggtgaccacgacgcgcaacatccgcgcgtcggtattcatggactggttcaccggtggcttgaactaccagatcgaccatcacctgttcccgctcgtgccgcgccacaacttgccaaaggtcaacgtgctcatcaagtcgctatgcaaggagttcgacatcccgttccacgagaccggcttctgggagggcatctacgaggtcgtggaccacctggcggacatcagcaaggaatttatcaccgagttcccagcgatgtaa

SEQ ID NO: 2 - protein, 459 aa, Phytium irregulare
MVDLKPGVKRLVSWKEIREHATPATAWIVIHHKVYDISKWDSHPGGSVMLTQAGEDATDAFAVFHPSSALKLLEQFYVGDVDETSKAEIEGEPASDEERARRERINEFIASYRRLRVKVKGMGLYDASALYYAWKLVSTFGIAVLSMAICFFFNSFAMYMVAGVIMGLFYQQSGWLAHDFLHNQVCENRTLGNLIGCLVGNAWQGFSMQWWKNKHNLHHAVPNLHSAKDEGFIGDPDIDTMPLLAWSKEMARKAFESAHGPFFIRNQAFLYFPLLLLARLSWLAQSFFYVFTEFSFGIFDKVEFDGPEKAGLIVHYIWQLAIPYFCNMSLFEGVAYFLMGQASCGLLLALVFSIGHNGMSVYERETKPDFWQLQVTTTRNIRASVFMDWFTGGLNYQIDHHLFPLVPRHNLPKVNVLIKSLCKEFDIPFHETGFWEGIYEVVDHLADISKEFITEFPAM

SEQ ID NO: 3 - DNA, 1320 bp, Thraustochytrium sp.
  CDS (1)..(1320): Delta-5-Desaturase
atgggcaagggcagcgagggccgcagcgcggcgcgcgagatgacggccgaggcgaacggcgacaagcggaaaacgattctgatcgagggcgtcctgtacgacgcgacgaactttaagcacccgggcggttcgatcatcaacttcttgaccgagggcgaggccggcgtggacgcgacgcaggcgtaccgcgagtttcatcagcggtccggcaaggccgacaagtacctcaagtcgctgccgaagctggatgcgtccaaggtggagtcgcggttctcggccaaagagcaggcgcggcgcgacgccatgacgcgcgactacgcggcctttcgcgaggagctcgtcgccgaggggtactttgacccgtcgatcccgcacatgatttaccgcgtcgtggagatcgtggcgctcttcgcgctctcgttctggctcatgtccaaggcctcgcccacctcgctcgtgctgggcgtggtgatgaacggcattgcgcagggccgctgcggctgggtcatgcacgagatgggccacgggtcgttcacgggcgtcatctggctcgacgaccggatgtgcgagttcttctacggcgtcggctgcggcatgagcgggcactactggaagaaccagcacagcaagcaccacgccgcgcccaaccgcctcgagcacgatgtcgatctcaacacgctgcccctggtcgcctttaacgagcgcgtcgtgcgcaaggtcaagccgggatcgctgctggcgctctggctgcgcgtgcaggcgtacctctttgcgcccgtctcgtgcctgctcatcggccttggctggacgctctacctgcacccgcgctacatgctgcgcaccaagcggcacatggagttcgtctggatcttcgcgcgctacattggctggttctcgctcatgggcgctctcggctactcgccgggcacctcggtcgggatgtacctgtgctcgttcggcctcggctgcatttacattttcctgcagttcgccgtcagccacacgcacctgccggtgaccaacccggaggaccagctgcactggctcgagtacgcggccgaccacacggtgaacattagcaccaagtcctggctcgtcacgtggtggatgtcgaacctgaactttcagatcgagcaccacctcttccccacggcgccgcagttccgcttcaaggaaatcagtcctcgcgtcgaggccctcttcaagcgccacaacctcccgtactacgacctgccctacacgagcgcggtctcgaccacctttgccaatctttattccgtcggccactcggtcggcgccgacaccaagaagcaggactga

SEQ ID NO: 4 - protein, 439 aa, Thraustochytrium sp.
MGKGSEGRSAAREMTAEANGDKRKTILIEGVLYDATNFKHPGGSIINFLTEGEAGVDATQAYREFHQRSGKADKYLKSLPKLDASKVESRFSAKEQARRDAMTRDYAAFREELVAEGYFDPSIPHMIYRVVEIVALFALSFWLMSKASPTSLVLGVVMNGIAQGRCGWVMHEMGHGSFTGVIWLDDRMCEFFYGVGCGMSGHYWKNQHSKHHAAPNRLEHDVDLNTLPLVAFNERVVRKVKPGSLLALWLRVQAYLFAPVSCLLIGLGWTLYLHPRYMLRTKRHMEFVWIFARYIGWFSLMGALGYSPGTSVGMYLCSFGLGCIYIFLQFAVSHTHLPVTNPEDQLHWLEYAADHTVNISTKSWLVTWWMSNLNFQIEHHLFPTAPQFRFKEISPRVEALFKRHNLPYYDLPYTSAVSTTFANLYSVGHSVGADTKKQD

SEQ ID NO: 5 - DNA, 1413 bp, Physcomitrella patens
  CDS (1)..(1413): Delta-5-Desaturase
atgtctgcgacatctgaagctgtgcggaatcacatcaagccaggaatcgttggcaggcccaatattgtgcttccaccattgagcgactttacagcgtcgaaacctacaagacttctcactaaaatccatggcaagtggtatgacttaacaaaattcgagaaacgtcatccgggaggaccagtggcgcttggtctggcgcgaggccgagacgccaccgttatgtttgagagtcaccatccattcaccaaccggaaaattttagatgccattttaatgaagtatgaaatcgatgcttcggacagcaaacacctacagactctggagcagcttcatggcgtacccgaacactctttcgaatggccgagtgcctttggcgaggccctgaaatttcaggttaaagagtatttcgaaggggagtctaagagacggaatatctccctccgagaagcgaccaaggcctctccttcgcggtgggttgagatcgcgatcttggctgtcctcttcttaagtacattccacgggttctttagaggtgattggagattcttgcttctgttcccgcttaccgcttggcttcttggagtgaatattttccatgacgcgactcacttcgccttctccgacaactggagatggaatgccttgatcccttacgctttcccttacttttcctctcccttttcttggtatcatcagcacaatataggtcaccacagctatccgaatgtttccgatcgggatccagatgtgctacaccactattggatgaagcgtgaacacagagacgtgaagtggttgcccattcacaagaatcagagcacttggtggtttatgctcttctggtggagtgtgtcggttgagtttggcttgacaaccatgcaggacctttggatgctgcagaccaatctttacaatgaggttgtgcctatgatggccattagcgggtcgaggaggctcaggcacattcttgggagagttttgacaattggaattattcacgcctggcctttcttcgtggtggagacttgggggaaggcctttgcattctctctcatcccgtatctatttttctcggttttgttcatgatgaacacgcagatcaaccatcttctaccccacacgacgcacgcagctgacgccgattggtacaagcatcaggtcatcaccgcccaggactttggagtgggaagcaagttctgccacctcttcagtggaggtttgaattatcaagtcattcaccacttgtttcccacagtgaaccactgtcatcttccgcaattgcaaccaattgttgctcggttatgcgagaaatacgacgttggttacacaactgctagaggttacgttcacgctattcaactacatcaccaacattcttcaaggttggctacaaagattgaacatgctgattaa

SEQ ID NO: 6 - protein, 470 aa, Physcomitrella patens
MSATSEAVRNHIKPGIVGRPNIVLPPLSDFTASKPTRLLTKIHGKWYDLTKFEKRHPGGPVALGLARGRDATVMFESHHPFTNRKILDAILMKYEIDASDSKHLQTLEQLHGVPEHSFEWPSAFGEALKFQVKEYFEGESKRRNISLREATKASPSRWVEIAILAVLFLSTFHGFFRGDWRFLLLFPLTAWLLGVNIFHDATHFAFSDNWRWNALIPYAFPYFSSPFSWYHQHNIGHHSYPNVSDRDPDVLHHYWMKREHRDVKWLPIHKNQSTWWFMLFWWSVSVEFGLTTMQDLWMLQTNLYNEVVPMMAISGSRRLRHILGRVLTIGIIHAWPFFVVETWGKAFAFSLIPYLFFSVLFMMNTQINHLLPHTTHAADADWYKHQVITAQDFGVGSKFCHLFSGGLNYQVIHHLFPTVNHCHLPQLQPIVARLCEKYDVGYTTARGYVHAIQLHHQHSSRLATKIEHAD

SEQ ID NO: 7 - DNA, 873 bp, Physcomitrella patens
  CDS (1)..(873): Delta-6-Elongase
atggaggtcgtggagagattctacggtgagttggatgggaaggtctcgcagggcgtgaatgcattgctgggtagttttggggtggagttgacggatacgcccactaccaaaggcttgcccctcgttgacagtcccacacccatcgtcctcggtgtttctgtatacttgactattgtcattggagggcttttgtggataaaggccagggatctgaaaccgcgcgcctcggagccatttttgctccaagctttggtgcttgtgcacaacctgttctgttttgcgctcagtctgtatatgtgcgtgggcatcgcttatcaggctattacctggcggtactctctctggggcaatgcatacaatcctaaacataaagagatggcgattctggtatacttgttctacatgtctaagtacgtggaattcatggataccgttatcatgatactgaagcgcagcaccaggcaaataagcttcctccacgtttatcatcattcttcaatttccctcatttggtgggctattgctcatcacgctcctggcggtgaagcatattggtctgcggctctgaactcaggagtgcatgttctcatgtatgcgtattacttcttggctgcctgccttcgaagtagcccaaagttaaaaaataagtaccttttttggggcaggtacttgacacaattccaaatgttccagtttatgctgaacttagtgcaggcttactacgacatgaaaacgaatgcgccatatccacaatggctgatcaagattttgttctactacatgatctcgttgctgtttcttttcggcaatttttacgtacaaaaatacatcaaaccctctgacggaaagcaaaagggagctaaaactgagtga

SEQ ID NO: 8 - protein, 290 aa, Physcomitrella patens
MEVVERFYGELDGKVSQGVNALLGSFGVELTDTPTTKGLPLVDSPTPIVLGVSVYLTIVIGGLLWIKARDLKPRASEPFLLQALVLVHNLFCFALSLYMCVGIAYQAITWRYSLWGNAYNPKHKEMAILVYLFYMSKYVEFMDTVIMILKRSTRQISFLHVYHHSSISLIWWAIAHHAPGGEAYWSAALNSGVHVLMYAYYFLAACLRSSPKLKNKYLFWGRYLTQFQMFQFMLNLVQAYYDMKTNAPYPQWLIKILFYYMISLLFLFGNFYVQKYIKPSDGKQKGAKTE

SEQ ID NO: 9 - DNA, 1086 bp, Phytophthora infestans
  CDS (1)..(1086): Omega-3-Desaturase
atggcgacgaaggaggcgtatgtgttccccactctgacggagatcaagcggtcgctacctaaagactgtttcgaggcttcggtgcctctgtcgctctactacaccgtgcgttgtctggtgatcgcggtggctctaaccttcggtctcaactacgctcgcgctctgcccgaggtcgagagcttctgggctctggacgccgcactctgcacgggctacatcttgctgcagggcatcgtgttctggggcttcttcacggtgggccacgatgccggccacggcgccttctcgcgctaccacctgctaaacttcgtggtgggcactttcatgcactcgctcatcctcacgcccttcgagtcgtggaagctcacgcaccgtcaccaccacaagaacacgggcaacattgaccgtgacgaggtcttctacccgcaacgcaaggccgacgaccacccgctgtctcgcaacctgattctggcgctcggggcagcgtggctcgcctatttggtcgagggcttccctcctcgtaaggtcaaccacttcaacccgttcgagcctctgttcgtgcgtcaggtgtcagctgtggtaatctctcttctcgcccacttcttcgtggccggactctccatctatctgagcctccagctgggccttaagacgatggcaatctactactatggacctgtttttgtgttcggcagcatgctggtcattaccaccttcctacaccacaatgatgaggagaccccatggtacgccgactcggagtggacgtacgtcaagggcaacctctcgtccgtggaccgatcgtacggcgcgctcattgacaacctgagccacaacatcggcacgcaccagatccaccaccttttccctatcattccgcactacaaactcaagaaagccactgcggccttccaccaggctttccctgagctcgtgcgcaagagcgacgagccaattatcaaggctttcttccgggttggacgtctctacgcaaactacggcgttgtggaccaggaggcgaagctcttcacgcttaaggaagccaaggcggcgaccgaggcggcggccaagaccaagtccacgtaa

SEQ ID NO: 10 - protein, 361 aa, Phytophthora infestans
MATKEAYVFPTLTEIKRSLPKDCFEASVPLSLYYTVRCLVIAVALTFGLNYARALPEVESFWALDAALCTGYILLQGIVFWGFFTVGHDAGHGAFSRYHLLNFVVGTFMHSLILTPFESWKLTHRHHHKNTGNIDRDEVFYPQRKADDHPLSRNLILALGAAWLAYLVEGFPPRKVNHFNPFEPLFVRQVSAVVISLLAHFFVAGLSIYLSLQLGLKTMAIYYYGPVFVFGSMLVITTFLHHNDEETPWYADSEWTYVKGNLSSVDRSYGALIDNLSHNIGTHQIHHLFPIIPHYKLKKATAAFHQAFPELVRKSDEPIIKAFFRVGRLYANYGVVDQEAKLFTLKEAKAATEAAAKTKST

SEQ ID NO: 11 - DNA, 1152 bp, Calendula officinalis
  CDS (1)..(1152): Delta-12-Desaturase
atgggtgcaggcggtcgaatgcaagatcccaccaacggtggcaacaaaaccgagcccgaaccaatccaacgggtcccacatgaaaaacccccattcacagttggagacatcaagaaagcgatcccacctcattgtttcaaccgatcggtaattcgttcattttcatacgtcttttacgacctcacaatcgcgtcaatcttgtactacattgccaacaattacatctctaccctccctagcccgctcgcctacgtggcatggcccgtttactgggccgtccaagggtgcgtcttaaccggggtgtgggtcatagcccacgaatgtggccatcatgcttttagcgaccaccaatggctcgatgacaccgtgggtctcgtcttgcactcgttcctactcgtgccctacttttcgtggaaatatagccaccgtaggcaccactcgaacacgggctcgatcgagcacgatgaggttttcgtcccgaagttgaaatcgggcgtccggtcaaccgcccggtacctaaacaacccaccgggccgaatcttgaccctactcgtaaccctaaccctcggttggcctctatacctcacgttcaacgtttcgggccgttactacgaccggttcgcgtgccatttcgacccgaatagcccgatctactcgaagcgcgaacgggctcaaatcttcatatccgacgccgggatcttagccgtagtcttcgtactcttccgactcgcaatgaccaaagggctcacgtgggtcctaaccatgtacggtggcccgttactcgtggtcaacggtttcctagtcttgatcacattcctacaacacactcacccttcgctcccgcactatgactcaaccgaatgggattggttacgtggggccctcaccacaatcgaccgtgattacgggatcctaaacaaagtgttccataacataaccgacactcacgtggcccaccatttgttctctacaatgcctcattaccatgcaatggaagccacgaaggtgatcaaaccgattttgggcgattattatcagtttgacgggacctcgatttttaaggcgatgtatcgggaaacaaaggagtgcatttatgttgataaggatgaggaggtgaaagatggtgtttattggtatcgtaataagatttaa

SEQ ID NO: 12 - protein, 383 aa, Calendula officinalis
MGAGGRMQDPTNGGNKTEPEPIQRVPHEKPPFTVGDIKKAIPPHCFNRSVIRSFSYVFYDLTIASILYYIANNYISTLPSPLAYVAWPVYWAVQGCVLTGVWVIAHECGHHAFSDHQWLDDTVGLVLHSFLLVPYFSWKYSHRRHHSNTGSIEHDEVFVPKLKSGVRSTARYLNNPPGRILTLLVTLTLGWPLYLTFNVSGRYYDRFACHFDPNSPIYSKRERAQIFISDAGILAVVFVLFRLAMTKGLTWVLTMYGGPLLVVNGFLVLITFLQHTHPSLPHYDSTEWDWLRGALTTIDRDYGILNKVFHNITDTHVAHHLFSTMPHYHAMEATKVIKPILGDYYQFDGTSIFKAMYRETKECIYVDKDEEVKDGVYWYRNKI

SEQ ID NO: 13 - DNA, 61 bp, Artificial sequence
  Primer
gaattcggcgcgccgagctcctcgagcaacggttccggcggtatagagttgggtaattcga

SEQ ID NO: 14 - DNA, 45 bp, Artificial sequence
  Primer
cccgggatcgatgccggcagatctccaccattttttggtggtgat

SEQ ID NO: 15 - DNA, 40 bp, Artificial sequence
  Primer
aggcctccatggcctgctttaatgagatatgcgagacgcc

SEQ ID NO: 16 - DNA, 32 bp, Artificial sequence
  Primer
cccgggccggacaatcagtaaattgaacggag

SEQ ID NO: 17 - DNA, 26 bp, Artificial sequence
  Primer
aggcctcaacggttccggcggtatag

SEQ ID NO: 18 - DNA, 60 bp, Artificial sequence
  Primer
cccggggttaacgctagcgggcccgatatcggatcccattttttggtggtgattggttct

SEQ ID NO: 19 - DNA, 31 bp, Artificial sequence
  Primer
aggcctcctgctttaatgagatatgcgagac

SEQ ID NO: 20 - DNA, 31 bp, Artificial sequence
  Primer
cccgggcggacaatcagtaaattgaacggag

SEQ ID NO: 21 - DNA, 28 bp, Artificial sequence
  Primer
aggcctcaacggttccggcggtatagag

SEQ ID NO: 22 - DNA, 50 bp, Artificial sequence
  Primer
aggccttctagactgcaggcggccgcccgcattttttggtggtgattggt

SEQ ID NO: 23 - DNA, 27 bp, Artificial sequence
  Primer
ggcctcctgctttaatgagatatgcga

SEQ ID NO: 24 - DNA, 51 bp, Artificial sequence
  Primer
aagcttggcgcgccgagctcgtcgacggacaatcagtaaattgaacggaga

SEQ ID NO: 25 - DNA, 30 bp, Artificial sequence
  Primer
agatctatggtggacctcaagcctggagtg

SEQ ID NO: 26 - DNA, 36 bp, Artificial sequence
  Primer
ccatggcccgggttacatcgctgggaactcggtgat

SEQ ID NO: 27 - DNA, 30 bp, Artificial sequence
  Primer
gggatccatgggcaagggcagcgagggccg

SEQ ID NO: 28 - DNA, 33 bp, Artificial sequence
  Primer
ggcgccgacaccaagaagcaggactgagatatc

SEQ ID NO: 29 - DNA, 36 bp, Artificial sequence
  Primer
gcggccgcatggaggtcgtggagagattctacggtg

SEQ ID NO: 30 - DNA, 31 bp, Artificial sequence
  Primer
gcaaaagggagctaaaactgagtgatctaga

SEQ ID NO: 31 - DNA, 31 bp, Artificial sequence
  Primer
gtcgatcaacggttccggcggtatagagttg

SEQ ID NO: 32 - DNA, 32 bp, Artificial sequence
  Primer
gtcgatcggacaatcagtaaattgaacggaga

SEQ ID NO: 33 - DNA, 28 bp, Artificial sequence
  Primer
agatctatgggtgcaggcggtcgaatgc

SEQ ID NO: 34 - DNA, 26 bp, Artificial sequence
  Primer
ccatggttaaatcttattacgatacc

(19) United States
(12) Patent Application Publication
(10) Pub. No.: US 2009/0172837 A1
(43) Pub. Date: Jul. 2, 2009

(54) PROCESS FOR PRODUCING ARACHIDONIC ACID AND/OR EICOSAPENTAENOIC ACID IN PLANTS

(76) Inventors: Michael Geiger, Ludwigshafen (DE); Jörg Bauer, Limburgerhof (DE); Petra Cirpus, Waldsee (DE)

Correspondence Address: CONNOLLY BOVE LODGE & HUTZ, LLP, P O BOX 2207, WILMINGTON, DE, 19899, US

(21) Appl. No.: 12/374,429

(22) PCT Filed: Jul. 11, 2007
(86) PCT No.: PCT/EP2007/057084

§ 371 Date: Jan. 20, 2009

(30) Foreign Application Priority Data
Jul. 21, 2006 (DE) 102006034313.1

(51) Int. Cl. C12N 15/82; C11B 1/00

(52) U.S. Cl. 800/281; 554/8

(57) ABSTRACT

The present invention relates to a process for the production of arachidonic acid (=APA) or eicosapentaenoic acid (=EPA) or arachidonic acid and eicosapentaenoic acid, advantageously in the seed of transgenic plants of the family Brassicaceae with a content of ARA or EPA or ARA and EPA of at least 3% by weight based on the total lipid content of the transgenic plant, by introducing, into the organism, nucleic acids which code polypeptides with Δ6-desaturase, Δ6-elongase and Δ5-desaturase activity, where, as the result of the enzymatic activity of the introduced enzymes, a fatty acid selected from the group consisting of the fatty acids oleic acid [C18:1^{Δ9}], linoleic acid [C18:2^{Δ9, 12}], α-linolenic acid [C18:3^{Δ6, 9, 12}], icosenoic acid (20:1^{Δ11}) and erucic acid [C22:1^{Δ11}] is reduced by at least 10% in comparison with the nontransgenic wild-type plant. Advantageously, further enzymes selected from the group of the enzymes (ω3-desaturases, Δ12-desaturases, Δ6-desaturases, Δ6-elongases, Δ5-desaturases, Δ5-elongases and/or Δ4-desaturases can be introduced into the plants.

The present invention relates to a process for the production of arachidonic acid (=ARA) or eicosapentaenoic acid (=EPA) or arachidonic acid and eicosapentaenoic acid, advantageously in the seed of transgenic plants of the family Brassicaceae with a content of ARA or EPA or ARA and EPA of at least 3% by weight based on the total lipid content of the transgenic plant, by introducing, into the organism, nucleic acids which code polypeptides with Δ6-desaturase, Δ6-elongase and Δ5-desaturase activity, where, as the result of the enzymatic activity of the introduced enzymes, a fatty acid selected from the group consisting of the fatty acids oleic acid [C18:1^{Δ9}], linoleic acid [C18:2^{Δ9, 12}], α-linolenic acid [C18:3^{Δ6, 9, 12}], icosenoic acid (20:1^{Δ11}) and erucic acid [C22:1^{Δ13}] is reduced by at least 10% in comparison with the nontransgenic wild-type plant. Advantageously, further enzymes selected from the group of the enzymes ω3-desaturases, Δ12-desaturases, Δ6-desaturases, Δ6-elongases, Δ5-desaturases, Δ5-elongases and/or Δ4-desaturases can be introduced into the plants.

The nucleic acid sequences are the sequences shown in SEQ ID NO: 1, SEQ ID NO: 3, SEQ ID NO: 5 or SEQ ID NO: 7. Preferably, further nucleic acid sequences which code polypeptides with ω3-desaturase or Δ12-desaturase activity are additionally introduced into the plant, in addition to these nucleic acid sequences, and also expressed simultaneously. Especially preferably, these are the nucleic acid sequences shown in SEQ ID NO: 9 and SEQ ID NO: 11.

These nucleic acid sequences can advantageously be expressed in the organism, if appropriate together with further nucleic acid sequences which code polypeptides of the biosynthesis of the fatty acid or lipid metabolism. Especially advantageous are nucleic acid sequences which code a Δ4-desaturase and/or Δ5-elongase activity. The oils, lipids or free fatty acids which comprise ARA and/or EPA are advantageously added, in quantities known to the skilled worker, to feedstuffs, foodstuffs, cosmetics or pharmaceuticals.

Lipid synthesis can be divided into two sections: the synthesis of fatty acids and their binding to sn-glycerol-3-phosphate, and the addition or modification of a polar head group. Usual lipids which are used in membranes comprise phospholipids, glycolipids, sphingolipids and phosphoglycerides. Fatty acid synthesis starts with the conversion of acetyl-CoA into malonyl-CoA by acetyl-CoA carboxylase or into acetyl-ACP by acetyl transacylase. After condensation reaction, these two product molecules together form acetoacetyl-ACP, which is converted via a series of condensation, reduction and dehydration reactions so that a saturated fatty acid molecule with the desired chain length is obtained. The production of the unsaturated fatty acids from these molecules is catalyzed by specific desaturases, either aerobically by means of molecular oxygen or anaerobically (regarding the fatty acid synthesis in microorganisms, see F. C. Neidhardt et al. (1996) E. coli and Salmonella. ASM Press: Washington, D.C., p. 612-636 and references cited therein; Lengeler et al. (Ed.) (1999) Biology of Procaryotes. Thieme: Stuttgart, New York, and the references therein, and Magnuson, K., et al. (1993) Microbiological Reviews 57:522-542 and the references therein). To undergo the further elongation steps, the resulting phospholipid-bound fatty acids must be returned to the fatty acid CoA ester pool from the phospholipids. This is made possible by acyl-CoA:lysophospholipid acyltransferases. Moreover, these enzymes are capable of transferring the elongated fatty acids from the CoA esters back to the phospholipids. If appropriate, this reaction sequence can be followed repeatedly.

Furthermore, fatty acids must subsequently be transported to various modification sites and incorporated into the triacylglycerol storage lipid. A further important step during lipid synthesis is the transfer of fatty acids to the polar head groups, for example by glycerol fatty acid acyltransferase (see Frentzenl, 1998, Lipid, 100(4-5):161-166).

With regard to publications on the biosynthesis of fatty acids in plants, desaturation, the lipid metabolism and the membrane transport of lipidic compounds, beta-oxidation, the modification of fatty acids and cofactors and the storage and assembly of triacylglycerol, including the references cited therein, see the following papers: Kinney, 1997, Genetic Engineering, Ed.: J K Setlow, 19:149-166; Ohlrogge and Browse, 1995, Plant Cell 7:957-970; Shanklin and Cahoon, 1998, Annu. Rev. Plant Physiol. Plant Mol. Biol. 49:611-641; Voelker, 1996, Genetic Engineering, Ed.: J K Setlow, 18:111-13; Gerhardt 1992, Prog. Lipid R. 31:397-417; Gühnemann-Schäfer & Kindl, 1995, Biochim. Biophys Acta 1256:181-186; Kunau et al., 1995, Prog. Lipid Res., 34:267-342; Stymne et al., 1993, in: Biochemistry and Molecular Biology of Membrane and Storage Lipids of Plants, Eds.: Murata and Somerville, Rockville, American Society of Plant Physiologists, 150-158, Murphy & Ross 1998, Plant Journal. 13(1):1-16.

In the text which follows, polyunsaturated fatty acids are referred to as PUFA, PUFAs, LCPUPA or LCPUFAs (poly unsaturated fatty acids, PUFA, long chain poly unsaturated fatty acids, LCPUFA. In particular, PUPA, PUFAs, LCPUFA and LCPUFAs are understood as meaning ARA, EPA and/or docosahexaenoic acid (=DHA).

Fatty acids and triacylglycerides have a multiplicity of applications in the food industry, in animal nutrition, in cosmetics and the pharmacological sector. Depending on whether they are free saturated or unsaturated fatty acids or else triacylglycerides with an elevated content of saturated or unsaturated fatty acids, they are suitable for very different applications. Polyunsaturated fatty acids such as linoleic and linolenic acid are essential for mammals since they cannot be produced by the latter. This is why polyunsaturated ω3-fatty acids and ω6-fatty acids are an important constituent of human and animal food. Thus, for example, lipids with unsaturated fatty acids, specifically with polyunsaturated fatty acids, are preferred in human nutrition. The polyunsaturated ω3-fatty acids are supposed to have a positive effect on the cholesterol level in the blood and thus on the prevention of heart disease. The risk of heart disease, strokes or hypertension can be reduced markedly by adding these ω3-fatty acids to the food (Shimikawa 2001, World Rev. Nutr. Diet. 88, 100-108).

ω3-fatty acids also have a positive effect on inflammatory, specifically on chronically inflammatory, processes in association with immunological diseases such as rheumatoid arthritis (Calder 2002, Proc. Nutr. Soc. 61, 345-358; Cleland and James 2000, J. Rheumatol. 27, 2305-2307). They are therefore added to foodstuffs, specifically to dietetic foodstuffs, or are employed in medicaments. ω6-fatty acids such as arachidonic acid tend to have a negative effect in connection with these rheumatological diseases. ARA, in turn, is advantageous and important in the development of newborn children.

ω3- and ω6-fatty acids are precursors of tissue hormones, known as eicosanoids, such as the prostaglandins, which are derived from dihomo-γ-linolenic acid, arachidonic acid and eicosapentaenoic acid, and of the thromboxanes and leukotrienes, which are derived from arachidonic acid and eicosapentaenoic acid.

Eicosanoids (known as the PG2 series) which are formed from the ω6-fatty acids, generally promote inflammatory reactions, while eicosanoids (known as the PG₃ series) from ω3-fatty acids have little or no proinflammatory effect.

Polyunsaturated long-chain ω3-fatty acids such as eicosapentaenoic acid (=EPA, C20:5^{Δ5, 8, 11, 14, 17}) or docosahexaenoic acid (=DHA, C22:6^{Δ4, 7, 10, 13, 16, 19}) are important components of human nutrition owing to their various roles in health aspects, including the development of the child brain, the functionality of the eyes, the synthesis of hormones and other signal substances, and the prevention of cardiovascular disorders, cancer and diabetes (Poulos, A Lipids 30:1-14, 1995; Horrocks, L A and Yeo Y K Pharmacol Res 40:211-225, 1999). There is therefore a demand for the production of polyunsaturated long-chain fatty acids, such as those mentioned above.

Owing to the present-day composition of human food, an addition of polyunsaturated ω3-fatty acids, which are preferentially found in fish oils, to the food is particularly important. Thus, for example, polyunsaturated fatty acids such as docosahexaenoic acid (=DHA, C22:6^{Δ4, 7, 10, 13, 16, 19}) or eicosapentaenoic acid (=EPA, C20:5^{Δ5, 8, 11, 14, 17}) are added to infant formula to improve the nutritional value. The unsaturated fatty acid DoA is supposed to have a positive effect on the development and maintenance of brain function. There is therefore a demand for the production of polyunsaturated long-chain fatty acids.

The various fatty acids and triglycerides are mainly obtained from microorganisms such as Mortierella or Schizochytrium or from oil-producing plants such as soybeans, oilseed rape, algae such as Crypthecodinium or Phaeodactylum and others, being obtained, as a rule, in the form of their triacylglycerides (=triglycerides=triglycerols). However, they can also be obtained from animals, for example, fish. The free fatty acids are advantageously prepared by hydrolysis. Very long-chain polyunsaturated fatty acids such as DHA, EPA, arachidonic acid (ARA, C20:4^{Δ5, 8, 11, 14}), dihomo-γ-linolenic acid (C20:3^{Δ8, 11, 14}) or docosapentaenoic acid (DPA, C22:5^{Δ7, 10, 13, 16, 19}) are, however, not synthesized in oil crops such as oilseed rape, soybeans, sunflowers and safflower. Conventional natural sources of these fatty acids are fish such as herring, salmon, sardine, redfish, eel, carp, trout, halibut, mackerel, zander or tuna, or algae.

Fatty acids from genera of the Brassicaceae family, such as Brassica napus or Brassica rapa, are well liked in the food, feedstuffs, cosmetics and/or pharmacological industries. The disadvantage of the oils from this family is that they comprise some fatty acids such as α-linolenic acid, icosenoic acid or erucic acid, which are rather undesirable, so that the oils cannot be used ad lib. Other fatty acids which are present in the oils, such as oleic acid, are of subordinate value as food additives.

Thus, longer-chain fatty acids such as icosenoic acid 20:1 and erucic acid 22:1 have been detected in the Brassicaceae family, in contrast to other plant families such as Linaceae, Poaceae or Leguminosac. For example, the erucic acid contents of Brassica carinata are 35-48%, of Brassica juncea 18-49%, of Brassica napus 45-54%, of Crambe abyssinica 55-60%, of Eruca sativa 34-47%, of Sinapis alba 33-51%, of Camelina sativa 3-5%, and of Raphanus sativa >22%.

Only very small amounts of erucic acid, if any, should be present in oils which are employed in human nutrition.

Advantageous oils, lipids and/or fatty acid compositions should have a very low content of fatty acids such as oleic acid, α-linolenic acid, icosenoic acid and/or erucic acid. Advantageously, the highest possible contents of fatty acids such as arachidonic acid and/or eicosapentaenoic acid should simultaneously be present. Moreover, the plants used for the production should be relatively simple to cultivate, and established processing procedures for the oils, lipids and/or fatty acid compositions which they comprise should be in existence. Moreover, the production process should be simple and economically advantageous. Moreover, the oils, lipids and/or fatty acid compositions of these plants should already have been used industrially for a prolonged period of time for the production of feeding stuffs, foodstuffs, cosmetics and/or pharmaceuticals.

It was therefore an object to develop a process for the production of large amounts of polyunsaturated fatty acids, specifically ARA, EPA and/or DHA, in the seed of transgenic plants while simultaneously reducing the contents of undesirable fatty acids.

This problem was solved by the process according to the invention for the production of arachidonic acid (=ARA) or eicosapentaenoic acid (=EPA) or arachidonic acid and eicosapentaenoic acid in transgenic plants of the Brassicaceae family with an ARA or EPA or ARA and EPA content of at least 3% by weight based on the total lipid content of the transgenic plant, characterized in that it comprises the following process steps:

a) introducing, into the useful plant, at least one nucleic acid sequence which codes for a Δ6-desaturase, and

b) introducing, into the useful plant, at least one nucleic acid sequence which codes for a Δ6-elongase, and

c) introducing, into the useful plant, at least one nucleic acid sequence which codes for a Δ5-desaturase, and

d) harvesting the useful plant,

where, as the result of the enzymatic activity of the enzymes introduced in steps a) to c), a fatty acid selected from the group consisting of the fatty acids oleic acid [C18:1^{Δ9}], linoleic acid [C18:2^{Δ9, 12}], α-linolenic acid [C18:3^{Δ6, 9, 12}], icosenoic acid [20:1^{Δ11}] and erucic acid [C22:1^{Δ11}] is reduced by at least 10%, 11%, 12%, 13%, 14% or 15%, advantageously by at least 16%, 17%, 18%, 19% or 20%, especially advantageously by at least 25%, 30%, 35% or 40%, in comparison with the nontransgenic wild-type plant.

The term wild-type plant is understood as meaning plants which contain the unmutated (unmodified) form of a gene or allele, and which predominantly occur in a population which lives under natural conditions. Wild-type also embraces so-called zero zygotes. The latter have been transformed with a gene, but have lost it again.

All the above data are percent by weight and relate to the fatty acid content in the corresponding wild-type plant.

The fatty acids produced in the process according to the invention advantageously comprise, besides arachidonic acid (=ARA) or eicosapentaenoic acid (=EPA) or arachidonic acid and eicosapentaenoic acid, yet further fatty acids such as polyunsaturated fatty acids, monounsaturated fatty acids and unsaturated fatty acids. Saturated fatty acids are advantageously converted only to a minor extent, or not at all, by the nucleic acids used in the process. By “to a minor extent” there is understood that the saturated fatty acids are converted with less than 5% of the activity, advantageously less than 3%, especially advantageously with less than 2%, very especially advantageously with less than 1, 0.5, 0.25 or 0.125%, in comparison with polyunsaturated fatty acids. These fatty acids which have been produced can be produced in the process as the single product, or else they can be present in a fatty acid mixture.

The nucleic acid sequences used in the process according to the invention are isolated nucleic acid sequences which code for polypeptides with Δ6-desaturase, Δ6-elongase and/or AS-desaturase activity.

Nucleic acid sequences which are advantageously used in the process according to the invention are nucleic acid sequences which code for polypeptides with Δ6-desaturase, Δ6-elongase or Δ5-desaturase activity, selected from the group consisting of:

a) a nucleic acid sequence with the sequence shown in SEQ ID NO: 1, SEQ ID NO, 3, SEQ ID NO: 5 or SEQ ID NO: 7, or

b) nucleic acid sequences which, as the result of the degeneracy of the genetic code, can be derived from the amino acid sequences shown in SEQ ID NO: 2, SEQ ID NO: 4, SEQ ID NO: 6 or SEQ ID NO: 8, or

c) derivatives of the nucleic acid sequence shown in SEQ ID NO: 1, SEQ ID NO: 3, SEQ ID NO: 5 or SEQ ID NO: 7, which code for polypeptides which have at least 40% identity at the amino acid level with SEQ ID NO: 2, SEQ ID NO: 4, SEQ ID NO: 6 or SEQ ID NO: 8 and which have a Δ6-desaturase, Δ6-elongase or Δ5-desaturase activity.

It is advantageous in the process to introduce, into the useful plants, further nucleic acid sequences which code for an ω3-desaturase or a Δ12-desaturase or an ω3-desaturase and a Δ12-desaturase.

A preferred embodiment of the process comprises that a nucleic acid sequence is additionally introduced, into the transgenic plant, which codes for polypeptides with ω3-desaturase activity, selected from the group consisting of:

a) a nucleic acid sequence with the sequence shown in SEQ ID NO: 9, or

b) nucleic acid sequences which, as the result of the degeneracy of the genetic code, can be derived from the amino acid sequence shown in SEQ ID NO: 10, or

c) derivatives of the nucleic acid sequence shown in SEQ ID NO: 9, which code for polypeptides which have at least 60% identity at the amino acid level with SEQ ID NO: 10 and which have an ω3-desaturase activity

In a further preferred embodiment, the process comprises that a nucleic acid sequence is additionally introduced, into the transgenic plant, which codes for polypeptides with Δ12-desaturase activity, selected from the group consisting of:

a) a nucleic acid sequence with the sequence shown in SEQ ID NO: 11, or

b) nucleic acid sequences which, as the result of the degeneracy of the genetic code, can be derived from the amino acid sequence shown in SEQ ID NO: 12, or

c) derivatives of the nucleic acid sequence shown in SEQ ID NO: 11, which code for polypeptides which have at least 60% identity at the amino acid level with SEQ ID NO: 10 and which have a Δ12-desaturase activity.

These abovementioned Δ12-desaturase sequences, alone or in combination with the ω3-desaturase sequences, can be used together with the nucleic acid sequences used in the process which code for Δ6-desaturases, Δ6-elongases and/or Δ5-desaturases.

The nucleic acids are advantageously expressed in vegetative or reproductive tissue.

The nucleic acid sequences used in the process not only lead to a reduction of the undesirable fatty acids, but also to an increase of the ARA or EPA or ARA and EPA content in the plants. Thus, it is possible to obtain, in the transgenic plants, an increase of the ARA or EPA or ARA and EPA content of up to more than 8%, advantageously up to more than 10%, 11%, 12%, 13%, 14%, 15%, 16%, 17%, 18%, 19% or 20%, especially advantageously up to more than 21%, 22%, 23%, 24% or 25%, based on the total lipid content of the plant, as the result of the process. The abovementioned percentages are percent by weight.

To further increase the yield in the above-described process for the production of oils and/or triglycerides with a polyunsaturated fatty acid content which is advantageously increased in comparison with oils and/or triglycerides from wild-type plants, especially of ARA, EPA or their mixtures, it may be advantageous to increase the amount of the starting material for the fatty acid synthesis. This can be achieved for example by introducing a nucleic acid which codes for a polypeptide with the activity of a Δ12-desaturase, and by coexpressing it in the organism.

This is especially advantageous in oil-producing plants of the genus Brassica, for example oilseed rape, turnip rape or Indian mustard, which have a high oleic acid content, but only a low linoleic acid content.

This is why, in a preferred embodiment of the present invention, a nucleic acid sequence which codes for a polypeptide with Δ12-desaturase activity is additionally introduced into the transgenic plant.

The Δ12-desaturases used in the process according to the invention advantageously convert oleic acid (C18:1^{Δ9}) into linoleic acid (C18:2^{Δ9, 12}) or C18:2^{Δ4, 9} into C18:3^{Δ6, 9, 12} (gamma-linolenic acid=GLA), the starting materials for the synthesis of ARA and/or EPA. The Δ12-desaturases used advantageously convert fatty acids which are bound to phospholipids or to CoA-fatty acid esters, advantageously those which are bound to CoA-fatty acid esters. If an elongation step has taken place beforehand, this advantageously leads to higher yields of synthetates, since elongation will, as a rule, take place on CoA-fatty acid esters, while desaturation predominantly takes place on the phospholipids or the triglycerides. An exchange between the CoA-fatty acid esters and the phospholipids or triglycerides, which would require a further, potentially limiting, enzymatic reaction, is therefore not required.

In a further advantageous embodiment of the process, all nucleic acid sequences will be introduced into the plants on a shared recombinant nucleic acid molecule, it being possible for each nucleic acid sequence to be under the control of its own promoter and it being possible for this own promoter to take the form of a seed-specific promoter.

However, it is not only the nucleic acids detailed in the sequence listing which can successfully be employed in the invention to carry out the conversion; rather, even sequences which deviate to a certain degree from these sequences and which code proteins with the essentially identical enzymatic activity can be employed. These take the form of nucleic acids which have a certain degree of identity or homology with the sequences specified in the sequence listing. An essentially identical enzymatic activity denotes proteins which have at least 20%, 30%, 40%, 50% or 60%, advantageously at least 70%, 80%, 90% or 95%, especially advantageously at least 96%, 97%, 98% or 99% of the enzymatic activity of the wild-type enzymes.

In order to determine the percentage of homology or identity of two amino acid sequences or of two nucleic acids, the sequences are written one under the other (for example, gaps may be introduced into the sequence of a protein or of a nucleic acid in order to generate optimal alignment with the other protein or the other nucleic acid). Then, the amino acid radicals or nucleotides at the corresponding amino acid positions or nucleotide positions are compared. If a position in a sequence is occupied by the same amino acid radical or the same nucleotide as the corresponding position in the other sequence, then the molecules are identical at this position. If different amino acids, which, however, have the same properties, for example two hydrophobic amino acids, occupy the same position, they are homologous or similar. The percentage of the identity or homology between the two sequences is a function of the number of positions which the sequences share (i.e. % homology=number of identical positions/total number of positions×100).

The identity was calculated over the entire amino acid or nucleic acid sequence region. To compare various sequences, the skilled worker has available a series of programs which are based on various algorithms. The algorithms of Needleman and Wunsch or Smith and Waterman give particularly reliable results. The program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970) and Smith and Waterman (Adv. Appl. Math. 2; 482-489 (1981)], which are part of the GCG software packet [Genetics Computer Group, 575 Science Drive, Madison, Wis., USA 53711 (1991)], were used to carry out the sequence comparisons. The sequence identity data given above in percent were determined over the entire sequence region using the program GAP with the following settings: Gap Weight: 50, Length Weight: 3, Average Match: 10.000 and Average Mismatch: 0.000. Unless otherwise specified, these settings were always used as standard settings for sequence comparisons.

The skilled worker will recognize that DNA sequence polymorphisms which lead to modifications of the amino acid sequence of the polypeptides used in the process may occur within a population. These natural variants usually cause a variance of from 1 to 5% in the nucleotide sequence of the Δ6-desaturase, Δ5-desaturase and/or Δ6-elongase gene. The scope of the invention is intended to comprise each and all of these nucleotide variation(s) and resulting amino acid polymorphisms in the Δ6-desaturase, Δ5-desaturase and/or Δ6-elongase which are the result of natural variation and which do not essentially modify the enzymatic activity.

Essential enzymatic activity of the Δ6-desaturase, Δ6-elongase or Δ5-desaturase used in the process according to the invention is understood as meaning that they retain an enzymatic activity of at least 10%, preferably of at least 20%, especially preferably of at least 30%, 40%, 50% or at least 60% and most preferably at least 70%, 80%, 90%, 95%, 96%, 97%, 98% or 99% in comparison with the proteins/enzymes coded by the sequence and its derivatives and that they are thus capable of participating in the metabolism of compounds which are required for the synthesis of fatty acids, fatty acid esters such as diacylglycerides and/or triacylglycerides in a plant or plant cell or in the transport of molecules across membranes.

Likewise, the scope of the invention comprises nucleic acid molecules which hybridize under stringent conditions with the complementary strand of the Δ12-desaturase, Δ6-desaturase, Δ5-desaturase, ω3-desaturase and/or Δ6-elongase nucleic acids used. The term “hybridizes under stringent conditions” as used in the present context is to describe hybridization and washing conditions under which nucleotide sequences with at least 60% homology to one another usually remain hybridized with one another. Conditions are preferably such that sequences with at least approximately 65%, 70%, 80% or 90%, preferably at least approximately 91%, 92%, 93%, 94% or 95%, and especially preferably at least approximately 96%, 97%, 98%, 99% or more homology to one another usually remain hybridized to one another. These stringent conditions are known to the skilled worker and described, for example, in Current Protocols in Molecular Biology, John Wiley & Sons, N.Y. (1989), 6.3.1-6.3.6.

A preferred, nonlimiting, example of stringent hybridization conditions is hybridizations in 6×sodium chloride/sodium citrate (=SSC) at approximately 45° C., followed by one or more washing steps in 0.2×SSC, 0.1% SDS at 50 to 65° C. The skilled worker knows that these hybridization conditions differ depending on the type of nucleic acid and, for example when organic solvents are present, regarding temperature and buffer concentration. Under “standard hybridization conditions”, for example, the hybridization temperature is, depending on the type of nucleic acid, between 42° C. and 58° C. in aqueous buffer with a concentration of 0.1 to 5×SSC (pH 7.2). If organic solvents, for example 50% formamide, are present in the abovementioned buffer, the temperature under standard conditions is approximately 42° C. Preferably the hybridization conditions for DNA:DNA hybrids, for example, are 0.1×SSC and 20° C. to 45° C., preferably 30° C. to 45° C. Preferably the hybridization conditions for DNA:RNA hybrids are, for example, 0.1×SSC and 30° C. to 55° C., preferably 45° C. to 55° C. The abovementioned hybridization temperatures are determined for a nucleic acid with approximately 100 bp (=base pairs) in length and with a G+C content of 50% in the absence of formamide. The skilled worker knows how to determine the required hybridization conditions on the basis of textbooks such as Sambrook et al., “Molecular Cloning”, Cold Spring Harbor Laboratory, 1989; Hames and Higgins (Eds.) 1985, “Nucleic Acids Hybridization: A Practical Approach”, IRL Press at Oxford University Press, Oxford; Brown (Ed.) 1991, “Essential Molecular Biology: A Practical Approach”, MIL Press at Oxford University Press, Oxford.

By introducing one or more nucleotide substitutions, additions or deletions into a nucleotide sequence, it is possible to generate an isolated nucleic acid molecule which codes a Δ12-desaturase, Δ6-desaturase, Δ5-desaturase, ω3-desaturase and/or Δ6-elongase with one or more amino acid substitutions, additions or deletions. Mutations can be introduced into one of the sequences by means of standard techniques, such as site-specific mutagenesis and PCR-mediated mutagenesis. It is preferred to generate conservative amino acid substitutions in one or more of the above nonessential amino acid radicals. In a “conservative amino acid substitution”, the amino acid radical is replaced by an amino acid radical with a similar side chain. Families of amino acid radicals with similar side chains have been defined in the art. These families comprise amino acids with basic side chains (for example lysine, arginine, histidine), acidic side chains (for example aspartic acid, glutamic acid), uncharged polar side chains (for example glycine, asparagine, glutamine, serine, threonine, tyrosine, cysteine), unpolar side chains (for example alanine, valine, leucine, isoleucine, proline, phenylalanine, methionine, tryptophan), beta-branched side chains (for example threonine, valine, isoleucine) and aromatic side chains (for example tyrosine, phenylalanine, tryptophan, histidine). A predicted nonessential amino acid radical in a Δ12-desaturase, Δ6-desaturase, Δ5-desaturase, ω3-desaturase and/or Δ6-elongase is thus preferably replaced by another amino acid radical from the same family of side chains. In another embodiment, the mutations can, alternatively, be introduced randomly over all or part of the sequence encoding the Δ12-desaturase, Δ6-desaturase, Δ5-desaturase, ω3-desaturase and/or Δ6-elongase, for example by saturation mutagenesis, and the resulting mutants can be screened by recombinant expression for the hereindescribed Δ12-desaturase, Δ6-desaturase, Δ5-desaturase, ω3-desaturase and/or Δ6-elongase activity in order to identify mutants which have retained the Δ12-desaturase, Δ6-desaturase, Δ5-desaturase, ω3-desaturase and/or Δ6-elongase activity.

The polyunsaturated fatty acids ARA or EPA or AKA and EPA produced in the process are advantageously bound as esters such as membrane lipids such as phospholipids or glycolipids and/or triacylglycerides, but may also occur in the organisms as free fatty acids or else bound in the form of other fatty acid esters. In this context, they may be present as “pure products” or else advantageously in the form of mixtures of various fatty acids or mixtures of different glycerides.

The various fatty acids which are bound in the triacylglycerides can be derived from short-chain fatty acids with 4 to 6 C atoms, medium-chain fatty acids with 8 to 12 C atoms or long-chain fatty acids with 14 to 24 C atoms, preferred are the long-chain fatty acids, especially preferred are the long-chain fatty acids LCPUFAs of C₁₈-, C₂₀- and/or C₂₂-fatty acids, very especially preferred are the long-chain fatty acids LCPUFAs of C₂₀- and/or C₂₂-fatty acids such as ARA, EPA or their combination.

The term “glyceride” is understood as meaning a glycerol which is esterified with one, two or three carboxylic acid residues (mono-, di- or triglyceride). The term “glyceride” is also understood as meaning a mixture of different glycerides. The glyceride, or the glyceride mixture, may comprise further additions, for example free fatty acids, antioxidants, proteins, carbohydrates, vitamins and/or other substances.

For the purposes of the process according to the invention a “glyceride” is furthermore understood as meaning derivatives derived from glycerol. This includes not only the above-described fatty acid glycerides, but also glycerophospholipids and glyceroglycolipids. Examples which may be mentioned by preference are the glycerophospholipids such as lecithin (pbosphatidylcholine), cardiolipin, phosphatidylglycerol, phosphatidylserine and alkylacylglycerophospholipids.

For the purposes of the invention, phospholipids are understood as meaning phosphatidylcholine, phosphatidylethanolamine, phosphatidylserine, phosphatidylglycerol and/or phosphatidylinositol, advantageously phosphatidylcholine.

The fatty acid esters produced in the process can be isolated in the form of an oil or lipid, for example in the form of compounds such as sphingolipids, phosphoglycerides, lipids, glycolipids such as glycosphingolipids, phospholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides or other fatty acid esters such as the acetyl-coenzyme A esters from the plants which were used for the preparation of the fatty acid esters. Advantageously, they are isolated in the form of their diacylglycerides, triacylglycerides and/or in the form of phospholipids such as phosphatidylcholine, especially preferably in the form of the triacylglycerides. In addition to these esters, the fatty acids produced in the process are also present in the plants as free fatty acids or bound in other compounds. As a rule, the various abovementioned compounds (fatty acid esters and free fatty acids) are present in the organisms with an approximate distribution of 80 to 90% by weight of triglycerides, 2 to 5% by weight of diglycerides, 5 to 10% by weight of monoglycerides, 1 to 5% by weight of free fatty acids, 2 to 8% by weight of phospholipids, the total of the various compounds amounting to 100% by weight.

In the method(s) according to the invention (for the purposes of the invention and the disclosure shown herein, the singular is to comprise the plural and vice versa), the LCPUFAs produced are produced in a content of at least 3, 5, 6, 7 or 8% by weight, advantageously at least 9, 10, 11, 12, 13, 14 or 15% by weight, preferably at least 16, 17, 18, 19 or 20% by weight, especially preferably at least 21, 22, 23, 24 or 25% by weight very especially preferably at least 26, 27, 28, 29 or 30% by weight based on the total fatty acids in the transgenic organisms, advantageously in the seeds of the transgenic plants. Here, C₁₈- and/or C₂₀-fatty acids which are present in the host organisms are advantageously converted into the corresponding products such as ARA, EPA or their mixtures, at the rate of at least 10%, advantageously at least 20%, especially advantageously at least 30%, very especially advantageously at least 40%. The fatty acids are advantageously produced in bound form.

Polyunsaturated C₂₀-fatty acids with four or five double bonds in the molecule are advantageously produced in the process in a content of all such fatty acids together of at least 15, 16, 17, 18, 19, or 20% by weight, advantageously at least 21, 22, 23, 24 or 25% by weight, especially advantageously at least 26, 27, 28, 29 or 30% by weight based on the total fatty acids in the seeds of the transgenic plants.

ARA is produced in the process according to the invention in a content of at least 3, 5, 6, 7, 8, 9 or 10% by weight advantageously at least 11, 12, 13, 14 or 15% by weight, preferably at least 16, 17, 18, 19 or 20% by weight, especially preferably at least 21, 22, 23, 24 or 25% by weight, most preferably at least 26% by weight, based on the total lipid content in the seeds of the transgenic plants.

EPA is produced in the process according to the invention in a content of at least 0.2, 0.3, 0.4, 0.5, 0.6, 0.7, 0.8, 0.9 or 1% by weight, advantageously at least 2, 3, 4 or 5% by weight, preferably at least 6, 7, 8, 9 or 10% by weight, especially preferably at least 11, 12, 13, 14 or 15% by weight and most preferably at least 16% by weight, based on the total lipid content in the seeds of transgenic plants.

It is possible, with the aid of the nucleic acids used in the process according to the invention, for these unsaturated fatty acids to be positioned at the sn1, sn2 and/or sn3 position of the triglycerides which have advantageously been produced. Since in the process according to the invention the starting compounds linoleic acid (C18:2) and linolenic acid (C18:3) pass through a plurality of reaction steps, the end product of the process, such as, for example, arachidonic acid (ARA) or eicosapentaenoic acid (EPA), are not obtained as absolutely pure products, small traces of the precursors are also always present in the end product. If, for example, both linoleic acid and linolenic acid are present in the starting organism, or the starting plants, the end products, such as ARA or EPA, are generally present as mixtures. It is advantageous that, in the end products ARA or EPA, only minor amounts of the in each case other end products should be present. This is why, in an EPA-comprising lipid and/or oil, less than 15, 14, 13, 12 or 11% by weight, advantageously less than 10, 9, 8, 7, 6 or 5% by weight, especially advantageously less than 4, 3, 2 or 1% by weight, of ARA should be present. This is also why in an ARA-comprising lipid and/or oil, less than 15, 14, 13, 12 or 11% by weight, advantageously less than 10, 9, 8, 7, 6 or 5% by weight, especially advantageously less than 4, 3, 2 or 1% by weight of EPA should be present.

The precursors should advantageously not amount to more than 20% by weight, preferably not to more than 15% by weight, especially preferably not to more than 10% by weight, very especially preferably not to more than 5% by weight, based on the amount of the end product in question. Advantageously, only ARA or EPA, bound or as free acids, are produced as end products in the process of the invention in a transgenic plant. If the compounds ARA and EPA are produced simultaneously, they are advantageously produced, in the plant, in a ratio of at least 1:6 (EPA:ARA), advantageously of at least 1:8, preferably of at least 1:10, especially preferably of at least 1:12.

Fatty acid esters or fatty acid mixtures produced by the process according to the invention advantageously comprise 6 to 15% of palmitic acid, 1 to 6% of stearic acid, 7-85% of oleic acid, 0.5 to 8% of vaccenic acid, 0.1 to 1% of arachic acid, 7 to 25% of saturated fatty acids, 8 to 85% of monounsaturated fatty acids and 60 to 85% of polyunsaturated fatty acids, in each case based on 100% and on the total fatty acid content of the organisms.

Moreover, the fatty acid esters or fatty acid mixtures which have been produced by the process of the invention advantageously comprise fatty acids selected from the group of the fatty acids erucic acid (13-docosaenoic acid), sterculic acid (9,10-methyleneoctadec-9-enoic acid), malvalic acid (8,9-methyleneheptadec-8-enoic acid), chaulmoogric acid (cyclopentenedodecanoic acid), furan fatty acid (9,12-epoxyoctadeca-9,11-dienoic acid), vernolic acid (9,10-epoxyoctadec-12-enoic acid), tariric acid (6-octadecynoic acid), 6-nonadecynoic acid, santalbic acid (t11-octadecen-9-ynoic acid), 6,9-octadecenynoic acid, pyrulic acid (t10-heptadecen-8-ynoic acid), crepenyninic acid (9-octadecen-12-ynoic acid), 13,14-dihydrooropheic acid, octadecen-13-ene-9,11-diynoic acid, petroselenic acid (cis-6-octadecenoic acid), 9c,12t-octadecadienoic acid, calendulic acid (8t10t12c-octadecatrienoic acid), catalpic acid (9t11t13c-octadecatrienoic acid), eleostearic acid (9c11t13t-octadecatrienoic acid), jacaric acid (8c10t12c-octadecatrienoic acid), punicic acid (9c11t13c-octadecatrienoic acid), parinaric acid (9c11t13t15c-octadecatetraenoic acid), pinolenic acid (all-cis-5,9,12-octadecatrienoic acid), laballenic acid (5,6-octadecadienallenic acid), ricinoleic acid (12-hydroxyoleic acid) and/or coriolic acid (13-hydroxy-9c,11t-octadecadienoic acid). The abovementioned fatty acids are, as a rule, advantageously only found in traces in the fatty acid esters or fatty acid mixtures produced by the process according to the invention, that is to say that, based on the total fatty acids, they occur to less than 30%, preferably to less than 25%, 24%, 23%, 22% or 21%, especially preferably to less than 20%, 15%, 10%, 9%, 8%, 7%, 6% or 5%, very especially preferably to less than 4%, 3%, 2% or 1%. In a further preferred form of the invention, these abovementioned fatty acids occur to less than 0.9%, 0.8%, 0.7%, 0.6% or 0.5%, especially preferably to less than 0.4%, 0.3%, 0.2%, 0.1%, based on the total fatty acids. The fatty acid esters or fatty acid mixtures produced by the process according to the invention advantageously comprise less than 0.1%, based on the total fatty acids, and/or no butyric acid, no cholesterol, no clupanodonic acid (=docosapentaenoic acid, C22:5^{Δ4, 8, 12, 15, 21}) and no nisinic acid (tetracosahexaenoic acid, C23:6^{Δ3, 8, 12, 15, 18, 21}).

Owing to the nucleic acid sequences according to the invention or nucleic acid sequences used in the process according to the invention, an increase in the yield of polyunsaturated fatty acids, mainly ARA and EPA, of at least 50, 80 or 100%, advantageously at least 150, 200 or 250%, especially advantageously at least 300, 400, 500, 600, 700, 800 or 900%, very especially advantageously at least 1000, 1100, 1200, 1300, 1400 or 1500% in comparison with the nontransgenic starting plant, for example a plant such as Brassica juncea or Brassica napus when compared by means of GC analysis may be achieved; see Examples.

The lipids and/or oils produced in the process according to the invention should advantageously have a high unsaturated, advantageously polyunsaturated, fatty acid content of at least 30, 40 or 50% by weight, advantageously at least 60, 70 or 80% by weight, based on the total fatty acid content in the seeds of the transgenic plants.

All saturated fatty acids together should advantageously only amount to a small quantity in the plants preferably used in the process according to the invention. In this context, a small amount is understood as meaning an amount of less than 15%, 14%, 13%, 12%, 11 % or 10%, preferably less than 9%, 8%, 7% or 6%, in units GC area.

Furthermore, the host plants which are advantageously used in the process and which comprise genes for the synthesis of the polyunsaturated fatty acids, which have been introduced, in the process, via different processes, should advantageously have a higher oil content than protein content in the seed, advantageous plants have an oil/protein content ratio of from 5:1, 4:1, 3:1, 2:1 or 1:1 In this context, the oil content based on the total weight of the seed should be in a range of 15-55%, advantageously between 25-50%, especially advantageously between 35-50%.

Host plants which are advantageous for the process are those which have a high oleic acid content, that means at least 40, 50, 60 or 70% by weight based on the total fatty acid content of the plant, in comparison with linoleic acid and/or linolenic acid in the lipids and/or oils, especially in the triglyceride, such as, for example, Brassica napus, Brassica alba, Brassica hirta, Brassica nigra, Brassica juncea or Brassica carinata.

Plants used for the process should advantageously have an erucic acid content of less than 2% by weight based on the total fatty acid content of the plant. Also, the content of saturated fatty acids C16:0 and/or C18:0 should advantageously be less than 19, 18, 17, 16, 15, 14, 13, 12, 11 or 10% by weight, advantageously less than 9, 8, 7, 6 or 5% by weight based on the total fatty acid content of the plant. Also, longer fatty acids such as C20:0 or C22:1 should advantageously not be present, or only in small amounts, advantageously in amounts of less than 4, 3, 2 or 1% by weight, advantageously less than 0.9, 0.8, 0.7, 0.6, 0.5, 0.4, 0.3, 0.2 or 0.1% by weight based on the total fatty acid content of the plant in the plants used in the process. Typically, C16:1 is not present as fatty acid, or only present in small amounts, in the plants used for the process according to the invention. Small amounts are advantageously understood as meaning fatty acid contents which are less than 4, 3, 2 or 1% by weight, advantageously less than 0.9, 0.8, 0.7, 0.6, 0.5, 0.4, 0.3, 0.2 or 0.1% by weight based on the total fatty acid content of the plant.

Chemically pure polyunsaturated fatty acids or fatty acid compositions can also be synthesized by the processes described above. To this end, the fatty acids or the fatty acid compositions are isolated from the plants, advantageously the seeds of the plants, in the known manner, for example via crushing the seeds, such as grinding, followed by extraction, distillation, crystallization, chromatography or a combination of these methods. These chemically pure fatty acids or fatty acid compositions are advantageous for applications in the food industry sector, the cosmetic sector and especially the pharmacological industry sector.

Plants which are suitable for the process according to the invention are, in principle, all those plants of the Brassicaceae family which are capable of synthesizing fatty acids, such as the genera Brassica, Camelina, Melanosinapis, Sinapis, Arabidopsis, for example the genera and species Brassica alba, Brassica carinata, Brassica hirta, Brassica napus, Brassica rapa ssp. [oilseed rape], Sinapis arvensis Brassica juncea, Brassica juncea var. juncea, Brassica juncea var. crispifolla, Brassica juncea var. foliosa, Brassica nigra, Brassica sinapioides, Camelina sativa, Melanosinapis communis [mustard], Brassica oleracea [fodder beet] or Arabidopsis thaliana.

It is advantageous for the described processes according to the invention to additionally introduce, into the plant, further nucleic acids which code for the enzymes of the fatty acid or lipid metabolism, in addition to the nucleic acids introduced in process steps (a) to (c) and in addition to the nucleic acid sequences which are optionally introduced and which code for the ω3-desaturases and/or for the Δ12-desaturases.

In principle, all genes of the fatty acid or lipid metabolism can be used in the process for the production of polyunsaturated fatty acids, advantageously in combination with the inventive Δ5-elongase(s), Δ6-elongase(s) and/or ω3-desaturases [for the purposes of the present application, the plural is understood as encompassing the singular and vice versa]. Genes of the fatty acid or lipid metabolism selected from the group consisting of acyl-CoA dehydrogenase(s), acyl-ACP [=acyl carrier protein) desaturase(s), acyl-ACP thioesterase(s), fatty acid acyl transferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene-oxide synthases, hydroperoxide lyases or fatty acid elongase(s) are advantageously used in combination with the Δ5-elongase, Δ6-elongase and/or ω3-desaturase. Genes selected from the group of the Δ4-desaturases, Δ5-desaturases, Δ6-desaturases, Δ8-desaturases, Δ9-desaturases, Δ12-desaturases, Δ6-elongases or Δ9-elongases are especially preferably used in combination with the above genes for the Δ5-elongase, Δ6-elongase and/or ω3-desaturase, it being possible to use individual genes or a plurality of genes in combination.

The nucleic acid sequences or their derivatives or homologs, which code for polypeptides which retain the enzymatic activity of the proteins coded by nucleic acid sequences, and which are used in the process according to the invention are, individually or in combination, advantageously cloned into expression constructs and used for the introduction into, and expression in, plants. Owing to their construction, these expression constructs make possible an advantageous optimal synthesis of the polyunsaturated fatty acids produced in the process according to the invention.

In a preferred embodiment, the process furthermore comprises the step of obtaining a transgenic plant which comprises the nucleic acid sequences used in the process, where the plant is transformed with a nucleic acid sequence which codes the Δ12-desaturase, Δ5-desaturase, Δ6-desaturase, Δ6-elongase and/or ω3-desaturase, a gene construct or a vector as described below, alone or in combination with further nucleic acid sequences which code proteins of the fatty acid or lipid metabolism. In a further preferred embodiment, this process furthermore comprises the step of obtaining the oils, lipids or free fatty acids from the seed of the plant.

In the case of plant cells, plant tissue or plant organs, “growing” is understood as meaning, for example, the cultivation on or in a nutrient medium, or of the intact plant on or in a substrate, for example in a hydroponic culture, potting compost or on arable land.

The invention furthermore relates to gene constructs which comprise the nucleic acid sequences according to the invention which code a Δ5-desaturase, Δ6-desaturase or Δ6-elongase, the nucleic acid being linked functionally with one or more regulatory signals. In addition, the gene construct may comprise further biosynthesis genes of the fatty acid or lipid metabolism selected from the group consisting of acyl-CoA dehydrogenase(s), acyl-ACP [=acyl carrier protein] desaturase(s), acyl-ACP thioesterase(s), fatty acid acyl transferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene-oxide synthases, hydroperoxide lyases or fatty acid elongase(s). Biosynthesis genes of the fatty acid or lipid metabolism selected from the group Δ12-desaturase or ω3-desaturase are advantageously additionally present.

The nucleic acid sequences used in the process which code proteins with Δ5-desaturase, Δ6-desaturase, Δ12-desaturase, ω3-desaturase or Δ6-elongase activity are advantageously introduced into the plant alone or, preferably, in combination with an expression cassette (=nucleic acid construct) which makes possible the expression of the nucleic acids in a plant. The nucleic acid construct can comprise more than one nucleic acid sequence with an enzymatic activity, for example, of a Δ5-desaturase, Δ6-desaturase, Δ12-desaturase, ω3-desaturase or Δ6-elongase.

To introduce the nucleic acids into the gene constructs, the nucleic acids used in the process are advantageously amplified and ligated in the known manner. Preferably, a procedure following the protocol for Pfu DNA polymerase or a Pfu/Taq DNA polymerase mixture is followed. The primers are selected taking into consideration the sequence to be amplified. The primers should expediently be chosen in such a way that the amplicon comprises the entire codogenic sequence from the start codon to the stop codon. After the amplification, the amplicon is expediently analyzed. For example, a gel-electrophoretic separation can be carried out, which is followed by a quantitative and a qualitative analysis. Thereafter, the amplicon can be purified following a standard protol (for example Qiagen). An aliquot of the purified amplicon is then available for the subsequent cloning step.

Suitable cloning vectors are generally known to the skilled worker. These include, in particular, vectors which are capable of replication in microbial systems, that is to say mainly vectors which ensure efficient cloning in yeasts or fungi and which make possible the stable transformation of plants. Those which must be mentioned in particular are various binary and cointegrated vector systems which are suitable for the T-DNA-mediated transformation. Such vector systems are, as a rule, characterized in that they comprise at least the vir genes required for the Agrobacterium-mediated transformation and the T-DNA-delimiting sequences (T-DNA border). These vector systems preferably also comprise further cis-regulatory regions such as promoters and terminator sequences and/or selection markers, by means of which suitably transformed organisms can be identified. While in the case of cointegrated vector systems vir genes and T-DNA sequences are arranged on the same vector, binary systems are based on at least two vectors, one of which bears vir genes, but no T-DNA, while a second one bears T-DNA, but no vir genes. Owing to this fact, the last-mentioned vectors are relatively small, easy to manipulate and capable of replication both in E. coli and in Agrobacterium. These binary vectors include vectors from the series pBIB-HYG, pPZP, pBecks, pGreen. In accordance with the invention, Bin19, pBI101, pBinAR, pGPTV and pCAMBIA are used by preference. An overview of the binary vectors and their use is found in Hellens et al, Trends in Plant Science (2000) 5, 446-451.

In order to prepare the vectors, the vectors can first be linearized with restriction endonuclease(s) and then modified enzymatically in a suitable manner. Thereafter, the vector is purified, and an aliquot is employed for the cloning step. In the cloning step, the enzymatically cleaved and, if appropriate, purified amplicon is ligated with vector fragments which have been prepared in a similar manner, using ligase. In this context, a particular nucleic acid construct, or vector or plasmid construct, can have one or more than one codogenic gene segments. The codogenic gene segments in these constructs are preferably linked functionally with regulatory sequences. The regulatory sequences include, in particular, plant sequences such as promoters and terminator sequences. The constructs can advantageously be stably propagated in microorganisms, in particular in E. coli and Agrobacterium tumefaciens, under selection conditions and make possible a transfer of heterologous DNA into plants or microorganisms.

The nucleic acids used in the process can be introduced into plants, advantageously using cloning vectors, and thus be used in the transformation of plants such as those which are published and cited therein: Plant Molecular Biology and Biotechnology (CRC Press, Boca Raton, Fla.), Chapter 6/7, p. 71-119 (1993); F. F. White, Vectors for Gene Transfer in Higher Plants; in: Transgenic Plants, Vol. 1, Engineering and Utilization, Eds.: Kung and R. Wu, Academic Press, 1993, 15-38; B. Jenes et al., Techniques for Gene Transfer, in: Transgenic Plants, Vol. 1, Engineering and Utilization, Eds.: Kung and R. Wu, Academic Press (1993), 128-143; Potrykus, Annu. Rev. Plant Physiol. Plant Molec. Biol. 42 (1991), 205-225. Thus, the nucleic acids and/or vectors used in the process can be used for the recombinant modification of a broad spectrum of plants so that the latter become better and/or more efficient PUFA producers.

A series of mechanisms by which a modification of the Δ5-desaturase, Δ6-desaturase, Δ12-desaturase, ω3-desaturase or Δ6-elongase protein is possible exists, so that the yield, production and/or production efficiency of the polyunsaturated fatty acids in a plant can be influenced directly owing to this modified protein. The number or activity of the Δ5-desaturase, Δ6-desaturase, Δ12-desaturase, ω3-desaturase or Δ6-elongase proteins or genes can be increased, so that greater amounts of the gene products and, ultimately, greater amounts of the compounds of the general formula I are produced. A de novo synthesis in a plant which has lacked the activity and ability to biosynthesize the compounds prior to introduction of the corresponding gene(s) I also possible. This applies analogously to the combination with further desaturases or elongases or further enzymes of the fatty acid and lipid metabolism. The use of various divergent sequences, i.e. sequences which differ at the DNA sequence level, may also be advantageous in this context, or else the use of promoters which make possible a different gene expression in the course of time, for example as a function of the degree of maturity of a seed or an oil-storing tissue.

The nucleic acid sequences used in the process are advantageously introduced into an expression cassette which makes possible the expression of the nucleic acids in plants.

In doing so, the nucleic acid sequences which code Δ5-desaturase, Δ6-desaturase, Δ12-desaturase, ω3-desaturase or Δ6-elongase are linked functionally with one or more regulatory signals, advantageously for enhancing gene expression. These regulatory sequences are intended to make possible the specific expression of the genes and proteins. Depending on the host organism, this may mean, for example, that the gene is expressed and/or overexpressed only after induction has taken place, or else that it is expressed and/or overexpressed immediately. For example, these regulatory sequences take the form of sequences to which inductors or repressors bind, thus controlling the expression of the nucleic acid. In addition to these novel regulatory sequences, or instead of these sequences, the natural regulatory elements of these sequences may still be present before the actual structural genes and, if appropriate, may have been genetically modified in such a way that their natural regulation is eliminated and the expression of the genes is enhanced. These modified promoters can also be positioned on their own before the natural gene in the form of part-sequences (=promoter with parts of the nucleic acid sequences used in accordance with the invention) in order to enhance the activity. Moreover, the gene construct may advantageously also comprise one or more what are known as enhancer sequences in operable linkage with the promoter, which make possible an enhanced expression of the nucleic acid sequence. Additional advantageous sequences, such as further regulatory elements or terminator sequences, may also be inserted at the 3′ end of the DNA sequences.

The Δ5-desaturase, Δ6-desaturase, Δ12-desaturase, ω3-desaturase or Δ6-elongase genes may be present in one or more copies of the expression cassette (=gene construct). Preferably, only one copy of the genes is present in each expression cassette. This gene construct, or the gene constructs, can be expressed together in the host plant. In this context, the gene construct(s) can be inserted in one or more vectors and be present in the cell in free form, or else be inserted in the genome. It is advantageous for the insertion of further genes in the host genome when the genes to be expressed are present together in one gene construct.

In this context, the regulatory sequences or factors can, as described above, preferably have a positive effect on the gene expression of the genes introduced, thus enhancing it. Thus, an enhancement of the regulatory elements, advantageously at the transcriptional level, may take place by using strong transcription signals such as promoters and/or enhancers. In addition, however, enhanced translation is also possible, for example by improving the stability of the mRNA.

In principle, it is possible to use all natural promoters together with their regulatory sequences, such as those mentioned above, for the novel process. It is also possible and advantageous to use synthetic promoters, either in addition or alone, in particular when they mediate seed-specific expression, such as those described in WO 99/16890.

In order to achieve a particularly high PUFA content, especially in transgenic plants, the PUFA biosynthesis genes should advantageously be expressed in oilseeds in a seed-specific manner. To this end, seed-specific promoters can be used, or those promoters which are active in the embryo and/or in the endosperm. In principle, seed-specific promoters can be isolated both from dicotyledonous and from monocotyledonous plants. Preferred promoters are listed hereinbelow: USP (=known seed protein) and vicilin (Vicia faba) [Bäumlein et al., Mol. Gen Genet., 1991, 225(3)], napin (oilseed rape) [U.S. Pat. No. 5,608,152], conlinin (linseed) [WO 02/102970], acyl carrier protein (oilseed rape) [U.S. Pat. No. 5,315,001 and WO 92/18634], oleosin (Arabidopsis thaliana) [WO 98/45461 and WO 93/20216], phaseolin (Phaseolus vulgaris) [U.S. Pat. No. 5,504,200], Bce4 [WO 91/13980], legumes B4 (LegB4 promoter) [Bäumlein et al., Plant J., 2, 2, 1992], Lpt2 and lpt1 (barley) [WO 95/15389 and WO95/23230], seed-specific promoters from rice, maize and wheat [WO 99/16890], Amy32b, Amy 6-6 and aleurain [U.S. Pat. No. 5,677,474], Bce4 (oilseed rape) [U.S. Pat. No. 5,530,149], glycinin (soybean) [EP 571 741], phosphoenol pyruvate carboxylase (soybean) [JP 06/62870], ADR12-2 (soybean) [WO 98/08962], isocitrate lyase (oilseed rape) [U.S. Pat. No. 5,689,040] or α-amylase (barley) [EP 781 849].

Plant gene expression can also be facilitated via a chemically inducible promoter (see a review in Gatz 1997, Annu. Rev. Plant Physiol. Plant Mol. Biol., 48:89-108). Chemically inducible promoters are particularly suitable when it is desired that gene expression should take place in a time-specific manner Examples of such promoters are a salicylic-acid-inducible promoter (WO 95/19443), a tetracycline-inducible promoter (Gatz et al. (1992) Plant J. 2, 397-404) and an ethanol-inducible promoter.

To ensure the stable integration of the biosynthesis genes into the transgenic plant over a plurality of generations, each of the nucleic acids which code Δ5-desaturase, Δ6-desaturase, Δ12-desaturase, ω3-desaturase or Δ6-elongase and which are used in the process should be expressed under the control of a separate promoter, preferably a promoter which differs from the other promoters, since repeating sequence motifs can lead to instability of the T-DNA, or to recombination events. In this context, the expression cassette is advantageously constructed in such a way that a promoter is followed by a suitable cleavage site, advantageously in a polylinker, for insertion of the nucleic acid to be expressed and, if appropriate, a terminator sequence is positioned behind the polylinker. This sequence is repeated several times, preferably three, four, five, six or seven times, so that up to seven genes can be combined in one construct and introduced into the transgenic plant in order to be expressed. Advantageously, the sequence is repeated up to four times. To express the nucleic acid sequences, the latter are inserted behind the promoter via a suitable cleavage site, for example in the polylinker. Advantageously, each nucleic acid sequence has its own promoter and, if appropriate, its own terminator sequence. Such advantageous constructs are disclosed, for example, in DE 101 02 337 or DE 101 02 338. However, it is also possible to insert a plurality of nucleic acid sequences behind a shared promoter and, if appropriate, before a shared terminator sequence. Here, the insertion site, or the sequence, of the inserted nucleic acids in the expression cassette is not of critical importance, that is to say a nucleic acid sequence can be inserted at the first or last position in the cassette without its expression being substantially influenced thereby. Advantageously, different promoters such as, for example, the USP, LegB4 or DC3 promoter, and different terminator sequences can be used in the expression cassette. However, it is also possible to use only one type of promoter in the cassette, which, however, may lead to undesired recombination events.

As described above, the transcription of the genes which have been introduced should advantageously be terminated by suitable terminator sequences at the 3′ end of the biosynthesis genes which have been introduced (behind the stop codon). An example of a sequence which can be used in this context is the OCS1 terminator sequence. As is the case with the promoters, different terminator sequences should be used for each gene.

As described above, the gene construct can also comprise further genes to be introduced into the plants. It is possible and advantageous to introduce into the host plants, and to express, regulatory genes such as genes for inductors, repressors or enzymes which, owing to their enzyme activity, engage in the regulation of one or more genes of a biosynthesis pathway. These genes can be of heterologous or of homologous origin.

Moreover, further biosynthesis genes of the fatty acid or lipid metabolism can advantageously be present in the nucleic acid construct, or gene construct; however, these genes can also be present on one or more further nucleic acid constructs. A biosynthesis gene of the fatty acid or lipid metabolism which is preferably chosen is a gene from the group consisting of acyl-CoA dehydrogenase(s), acyl-ACP [=acyl carrier protein] desaturase(s), acyl-ACP thioesterase(s), fatty acid acyl transferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenase(s), lipoxygenase(s), triacylglycerol lipase(s), allene-oxide synthase(s), hydroperoxide lyase(s) or fatty acid elongase(s) or combinations thereof.

Especially advantageous nucleic acid sequences are biosynthesis genes of the fatty acid or lipid metabolism selected from the group of the acyl-CoA:lysophospholipid acyltransferase, Δ8-desaturase, Δ4-desaturase, Δ9-desaturase, Δ5-elongase and/or Δ9-elongase.

In this context, the abovementioned nucleic acids or genes can be cloned into expression cassettes, like those mentioned above, in combination with other elongases and desaturases and used for transforming plants with the aid of Agrobacterium.

Here, the regulatory sequences or factors can, as described above, preferably have a positive effect on, and thus enhance, the gene expression of the genes which have been introduced. Thus, enhancement of the regulatory elements can advantageously take place at the transcriptional level by using strong transcription signals such as promoters and/or enhancers. However, an enhanced translation is also possible, for example by improving the stability of the mRNA. In principle, the expression cassettes can be used directly for introduction into the plants or else be introduced into a vector.

These advantageous vectors, preferably expression vectors, comprise the nucleic acids which code the Δ5-desaturase, Δ6-desaturase, Δ12-desaturase, ω3-desaturase or Δ6-elongase and which are used in the process, or else a nucleic acid construct which comprises the nucleic acid used either alone or in combination with further biosynthesis genes of the fatty acid or lipid metabolism such as the acyl-CoA:lysophospholipid acyltransferases, Δ8-desaturases, Δ9-desaturases, Δ4-desaturases, Δ5-elongases and/or Δ9-elongases.

As used in the present context, the term “vector” refers to a nucleic acid molecule which is capable of transporting another nucleic acid to which it is bound. One type of vector is a “plasmid”, a circular double-stranded DNA loop into which additional DNA segments can be ligated. A further type of vector is a viral vector, it being possible for additional DNA segments to be ligated into the viral genome. Certain vectors are capable of autonomous replication in a host cell into which they have been introduced (for example bacterial vectors with bacterial replication origin). Other vectors are advantageously integrated into the genome of a host cell when they are introduced into the host cell, and thus replicate together with the host genome. Moreover, certain vectors can govern the expression of genes with which they are in operable linkage. These vectors are referred to in the present context as “expression vectors”. Usually, expression vectors which are suitable for DNA recombination techniques take the form of plasmids. In the present description, “plasmid” and “vector” can be used exchangeably since the plasmid is the form of vector which is most frequently used. However, the invention is also intended to cover other forms of expression vectors, such as viral vectors, which exert similar functions. Furthermore, the term “vector” is also intended to encompass other vectors with which the skilled worker is familiar, such as phages, viruses such as SV40, CMV, TMV, transposons, IS elements, phasmids, phagemids, cosmids, linear or circular DNA.

The recombinant expression vectors advantageously used in the process comprise the nucleic acids or the described gene construct used in accordance with the invention in a form which is suitable for expressing the nucleic acids used in a host cell, which means that the recombinant expression vectors comprise one or more regulatory sequences, selected on the basis of the host cells used for the expression, which regulatory sequence(s) is/are linked functionally with the nucleic acid sequence to be expressed. In a recombinant expression vector, “linked functionally” or “in operable linkage” means that the nucleotide sequence of interest is bound to the regulatory sequence(s) in such a way that the expression of the nucleotide sequence is possible and they are bound to each other in such a way that both sequences carry out the predicted function which is ascribed to the sequence (for example in an in-vitro transcription/translation system, or in a host cell if the vector is introduced into the host cell).

The term “regulatory sequence” is intended to comprise promoters, enhancers and other expression control elements (for example polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnology, CRC Press, Boca Raton, Fla., Eds.: Glick and Thompson, Chapter 7, 89-108, including the references cited therein. Regulatory sequences comprise those which govern the constitutive expression of a nucleotide sequence in many types of host cell and those which govern the direct expression of the nucleotide sequence only in specific host cells under specific conditions. The skilled worker knows that the design of the expression vector can depend on factors such as the choice of host cell to be transformed, the desired expression level of the protein and the like.

In a further embodiment of the process, the Δ12-desaturases, Δ6-desaturases, ω3-desaturases, Δ6-elongases and/or Δ5-desaturases can be expressed in single-celled plant cells (such as algae), see Falciatore et al., 1999, Marine Biotechnology 1 (3):239-251 and references cited therein, and in plant cells from higher plants (for example spermatophytes such as arable crops). Examples of plant expression vectors comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Eds.: Kung and R. Wu, Academic Press, 1993, p. 15-38.

A plant expression cassette preferably comprises regulatory sequences which are capable of governing the expression of genes in plant cells and which are linked functionally so that each sequence can fulfill its function, such as transcriptional termination, for example polyadenylation signals. Preferred polyadenylation signals are those which are derived from Agrobacterium tumefaciens T-DNA, such as gene 3 of the Ti plasmid pTiACH5 (Gielen et al., EMBO J. 3 (1984) 835 et seq.), which is known as octopine synthase, or functional equivalents thereof, but all other terminator sequences which are functionally active in plants are also suitable.

Since the regulation of plant gene expression is very often not limited to the transcriptional level, a plant expression cassette preferably comprises other sequences which are linked functionally, such as translation enhancers, for example the overdrive sequence, which enhances the tobacco mosaic virus 5′-untranslated leader sequence, which increases the protein/RNA ratio (Gallie et al., 1987, Nucl. Acids Research 15:8693-8711).

As described above, the gene to be expressed must be linked functionally with a suitable promoter which triggers gene expression with the correct timing or in a cell- or tissue-specific manner. Utilizable promoters are constitutive promoters (Benfey et al., EMBO J. 8 (1989) 2195-2202), such as those which are derived from plant viruses, such as 35S CaMV (Franck et al., Cell 21 (1980) 285-294), 19S CaMV (see also U.S. Pat. No. 5,352,605 and WO 84/02913), or constitutive plant promoters, such as the promoter of the Rubisco small subunit, which is described in U.S. Pat. No. 4,962,028.

As described above, plant gene expression can also be achieved via a chemically inducible promoter (see a review in Gatz 1997, Annu. Rev. Plant Physiol. Plant Mol. Biol., 48:89-108). Chemically inducible promoters are particularly suitable when it is desired that the gene expression takes place in a time-specific manner. Examples of such promoters are a salicylic-acid-inducible promoter (WO 95/19443), a tetracycline-inducible promoter (Gatz et al. (1992) Plant J. 2, 397-404) and an ethanol-inducible promoter.

Promoters which respond to biotic or abiotic stress conditions are also suitable, for example the pathogen-induced PRPI gene promoter (Ward et al., Plant. Mol. Biol. 22 (1993) 361-366), the heat-inducible tomato hsp80 promoter (U.S. Pat. No. 5,187,267), the chill-inducible potato alpha-amylase promoter (WO 96/12814) or the wound-inducible pinII promoter (EP-A-0 375 091).

Especially preferred are those promoters which bring about the gene expression in tissues and organs in which the biosynthesis of fatty acids, lipids and oils takes place, in seed cells, such as cells of the endosperm and of the developing embryo. Suitable promoters are the oilseed rape napin promoter (U.S. Pat. No. 5,608,152), the linseed Conlinin promoter (WO 02/102970), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legume B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley lpt2 or lpt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890.

Other promoters which are also particularly suitable are those which bring about the plastid-specific expression, since plastids constitute the compartment in which precursors and some end products of lipid biosynthesis are synthesized. Suitable promoters are the viral RNA polymerase promoter, described in WO 95/16783 and WO 97/06250, and the Arabidopsis clpP promoter, described in WO 99/46394.

Advantageous regulatory sequences for the new process are present for example in promoters such as the plant promoters CaMV/35S [Franck et al., Cell 21 (1980) 285-294)], PRP 1 [Ward et al., Plant Mol. Biol. 22 (1993)], SSU, OCS, lib4, usp, STLS1, B33, nos or in the ubiquitin or phaseolin promoter. Also advantageous in this context are inducible promoters, such as the promoters described in EP-A-0 388 186 (benzylsulfonamide-inducible), Plant J. 2, 1992:397-404 (Gatz et al., tetracyclin-inducible), EP-A-0 335 528 (abscisic-acid-inducible) or WO 93/21334 (ethanol- or cyclohexenol-inducible). Further suitable plant promoters are the promoter of cytosolic FBPase or the ST-LSI promoter from potato (Stockhaus et al., EMBO J. 8, 1989, 2445), the phosphoribosyl-pyrophosphate amidotransferase promoter from Glycine max (Genbank accession No. U87999) or the node-specific promoter described in EP-A-0 249 676. Especially advantageous promoters are promoters which enable the expression in tissues which are involved in the biosynthesis of fatty acids. Very especially advantageous are seed-specific promoters such as the USP promoter in accordance with the practice, but also other promoters such as the LeB4, DC3, phaseolin or napin promoters. Further especially advantageous promoters are seed-specific promoters which can be used for monocotyledonous or dicotyledonous plants and which are described in U.S. Pat. No. 5,608,152 (napin promoter from oilseed rape), WO 98/45461 (oleosin promoter from Arabidopsis), U.S. Pat. No. 5,504,200 (phaseolin promoter from Phaseolus vulgaris), WO 91/13980 (Bce4 promoter from Brassica), by Baeumlein et al., Plant J., 2, 2, 1992:233-239 (LeB4 promoter from a legume), these promoters being suitable for dicots. The following promoters are suitable for example for monocots: lpt-2 or lpt-1 promoter from barley (WO 95/15389 and WO 95/23230), hordein promoter from barley and other promoters which are suitable and which are described in WO 99/16890.

In principle, it is possible to use all natural promoters together with their regulatory sequences, such as those mentioned above, for the novel process. Likewise, it is possible and advantageous to use synthetic promoters, either additionally or alone, especially when they mediate a seed-specific expression, such as, for example, as described in WO 99/16890.

To obtain a particularly high PUFA content especially in transgenic plants, the PUFA biosynthesis genes should advantageously be expressed in a seed-specific manner in oilseed crops. To this end, it is possible to use seed-specific promoters or those promoters which are active in the embryo and/or in the endosperm. In principle, seed-specific promoters can be isolated both from dicotyledonous and from monocotyledonous plants. Such advantageous promoters are detailed further above, for example the USP, Vicilin, Napin, Oleosin, Phaseolin, Bce4, LegB4, Lpt2, lpt1, Amy32b, Amy 6-6, Aleurain or Bce4 promoter.

Moreover, chemically inducible promoters are also advantageously useful in the process according to the invention.

Further advantageous promoters which are advantageously suitable for expression in soybean are the promoters of the β-conglycinin α-subunit, of the β-conglycinin β-subunit, of the Kunitz trypsin inhibitor, of annexin, of glysinin, of albumin 2S, of legumin A1, of legumin Δ2 and that of BD30.

Especially advantageous promoters are the USP, LegB4, Fad3, SBP, DC-3 or cruciferin820 promoter.

Advantageous regulatory sequences which are used for the expression of the nucleic acid sequences used in the process according to the invention are terminators for the expression advantageously in soybean are Leg2A3′, Kti3′, Phas3′, BD30 3′ or AIS3′.

Especially advantageous terminators are the A7T, OCS, LeB3T or cat terminator.

To ensure a stable integration of the biosynthetic genes in the transgenic plant over several generations, each of the nucleic acids used in the process and which code Δ12-desaturases, ω3-desaturases, Δ6-desaturases, Δ6-elongases and/or Δ5-desaturases should, as described above, be under the control of its own promoter, preferably of a different promoter, since repeating sequence motifs can lead to instability of the T-DNA, or to recombination events. As described above, the gene construct can also comprise further genes which are to be introduced into the plant.

In this context, the regulatory sequences or factors used advantageously for the expression of the nucleic acids used in the process according to the invention can, as described above, preferably have a positive effect on, and thereby enhance, the gene expression of the genes introduced.

These advantageous vectors, preferably expression vectors, comprise the nucleic acids used in the process which code the Δ12-desaturases, ω3-desaturases, Δ6-desaturases, Δ6-elongases and/or Δ5-desaturases, or a nucleic acid construct which the used nucleic acid alone or in combination with further biosynthesis genes of the fatty acid or lipid metabolism such as the acyl-CoA:lysophospholipid acyltransferases, ω3-desaturases, Δ4-desaturases, Δ5-desaturases, Δ6-desaturases, Δ8-desaturases, Δ9-desaturases, Δ12-desaturases, ω3-desaturases, Δ5-elongases, Δ6-elongases and/or Δ9-elongases.

As described and used in the present context, the term “vector” refers to a nucleic acid molecule which is capable of transporting another nucleic acid to which it is bound.

The recombinant expression vectors used can be designed for expressing Δ12-desaturases, ω3-desaturases, Δ6-desaturases, Δ6-elongases and/or Δ5-desaturases, in prokaryotic or eukaryotic cells. This is advantageous since, for the sake of simplicity, intermediate steps of the vector construction are frequently carried out in microorganisms. For example, the Δ12-desaturase, ω3-desaturase, Δ6-desaturase, Δ6-elongase and/or Δ5-desaturase genes can be expressed in bacterial cells, insect cells (using baculovirus expression vectors), yeast cells and other fungal cells (see Romanos, M. A., et al. (1992) “Foreign gene expression in yeast: a review”, Yeast 8:423-488; van den Hondel, C. A. M. J. J., et al. (1991) “Heterologous gene expression in filamentous fungi”, in: More Gene Manipulations in Fungi, J. W. Bennet & L. L. Lasure, Ed., pp. 396-428: Academic Press: San Diego; and van den Hondel, C. A. M. J. J., & Punt, P. J. (1991) “Gene transfer systems and vector development for filamentous fungi, in: Applied Molecular Genetics of Fungi, Peberdy, J. F., et al., Ed., pp. 1-28, Cambridge University Press: Cambridge), algae (Falciatore et al., 1999, Marine Biotechnology. 1, 3:239-25 1), ciliates of the types: Holotrichia, Peritrichia, Spirotrichia, Suctoria, Tetrahymena, Paramecium, Colpidium, Glaucoma, Platyophrya, Potomacus, Desaturaseudocohnilembus, Euplotes, Engelmaniella and Stylonychia, in particular the genus Stylonychia lemnae, using vectors following a transformation process as described in WO 98/01572, and preferably in cells of multi-celled plants (see Schmidt, R. and Willmitzer, L. (1988) “High efficiency Agrobacterium tumefaciens-mediated transformation of Arabidopsis thaliana leaf and cotyledon explants” Plant Cell Rep.: 583-586; Plant Molecular Biology and Biotechnology, C Press, Boca Raton, Fla., chapter 6/7, pp. 71-119 (1993); F. F. White, B. Jenes et al., Techniques for Gene Transfer, in: Transgenic Plants, Vol. 1, Engineering and Utilization, Ed.: Kung and R. Wu, Academic Press (1993), 128-43; Potrykus, Annu. Rev. Plant Physiol. Plant Molec. Biol. 42 (1991), 205-225 (and references cited therein)). Suitable host cells are furthermore discussed in Goeddel, Gene Expression Technology: Methods in Enzymology 185, Academic Press, San Diego, Calif. (1990). As an alternative, the recombinant expression vector can be transcribed and translated in vitro, for example using T7-promoter regulatory sequences and T7-polymerase.

In most cases, the expression of proteins in prokaryotes, advantageously for the simple detection of the enzyme activity for example for detecting the desaturase or elongase activity, is performed using vectors comprising constitutive or inducible promoters which control the expression of fusion or nonfusion proteins. Examples of typical fusion expression vectors are pGEX (Pharmacia Biotech Inc; Smith, D. B., and Johnson, K. S. (1988) Gene 67:31-40), pMAL (New England Biolabs, Beverly, Mass.) and pRITS (Pharmacia, Piscataway, N.J.), where glutathione S-transferase (GST), maltose-E-binding protein and protein A, respectively, are fused with the recombinant target protein.

Examples of suitable inducible nonfusion E. coli expression vectors are, inter alia, pTrc (Amann et al. (1988) Gene 69:301-315) and pET 11d (Studier et al., Gene Expression Technology: Methods in Enzymology 185, Academic Press, San Diego, Calif. (1990) 60-89). The target gene expression of the pTrc vector is based on the transcription from a hybrid trp-lac fusion promoter by host RNA polyerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS 174 (DE3) from a resident λ-prophage which harbors a T7 gn1 gene under the transcriptional control of the lacUV 5 promoter.

The skilled worker is familiar with other vectors which are suitable in prokaryotic organisms, these vectors are, for example E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, λgtl1 or pBdCl, in Streptomyces plJ101, plJ364, plJ702 or plJ361, in Bacillus pUB110, pC194 or pBD214, in Corynebacterium pSA77 or pAJ667.

In a further embodiment, the expression vector is a yeast expression vector. Examples of vectors for expression in the yeast S. cerevisiae comprise pYeDesaturasec1 (Baldari et al. (1987) Embo J. 6:229-234), pMFa (Kurjan and Herskowitz (1982) Cell 30:933-943), pJRY88 (Schultz et al. (1987) Gene 54-113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed. pp. 1-28, Cambridge University Press: Cambridge, or in: More Gene Manipulations in Fungi [J. W. Bennett & L. L. Lasure, Ed., pp. 396-428: Academic Press: San Diego]. Further suitable yeast vectors are, for example, pAG-1, YEp6, YEp13 or pEMBLYe23.

The abovementioned vectors are only a small overview of possible suitable vectors. Further plasmids are known to the skilled worker and are described, for example, in: Cloning Vectors (Ed., Pouwels, P. H., et al., Elsevier, Amsterdam-New York-Oxford, 1985, ISBN 0 444 904018). Further suitable expression systems for prokaryotic and eukaryotic cells, see the chapters 16 and 17 of Sambrook, J., Fritsch, E. F., and Maniatis, T., Molecular Cloning: A Laboratory Manual, 2^{nd} edition, Cold Spring Harbor Laboratory, Cold Spring Harbor Laboratory Press, Cold Spring Harbor, N.Y., 1989.

A plant expression cassette preferably comprises regulatory sequences which are capable of governing the expression of genes in plant cells and which are linked functionally so that each sequence can fulfill its function, such as transcriptional termination, for example polyadenylation signals. Preferred polyadenylation signals are those which are derived from Agrobacterium tumefaciens T-DNA, such as gene 3 of the Ti plasmid pTiACH5 (Gielen et al., EMBO J. 3 (1984) 835 et seq.), which is known as octopine synthase, or functional equivalents thereof, but all other terminator sequences which are functionally active in plants are also suitable.

Since plant gene expression is very often not limited to the transcriptional level, a plant expression cassette preferably comprises other sequences which are linked functionally, such as translation enhancers, for example the overdrive sequence, which enhances the tobacco mosaic virus 5′-untranslated leader sequence, which increases the protein/RNA ratio (Gallie et al., 1987, Nucl. Acids Research 15:8693-8711).

As described above, plant gene expression must be linked operably with a suitable promoter which triggers gene expression with the correct planning or in a cell- or tissue-specific manner. Utilizable promoters are constitutive promoters (Benfey et al., EMBO J. 8 (1989) 2195-2202), such as those which are derived from plant viruses, such as 35S CaMV (Franck et al., Cell 21 (1980) 285-294), 19S CaMV (see also U.S. Pat. No. 5,352,605 and WO 84/02913), or plant promoters such as the promoter of the Rubisco small subunit, which is described in U.S. Pat. No. 4,962,028.

Other sequences which are preferred for use in the operable linkage in plant gene expression cassettes are targeting sequences, which are required for targeting the gene product into its corresponding cell compartment (for an overview, see Kermode, Crit. Rev. Plant Sci. 15, 4 (1996) 285-423 and references cited therein), for example into the vacuole, the nucleus, all types of plastids such as amyloplasts, chloroplasts, chromoplasts, the extracellular space, the mitochondria, the endoplasmic reticulum, oil bodies, peroxisomes and other compartments of plant cells.

As described above, plant gene expression can also be achieved via a chemically inducible promoter (see a review in Gatz 1997, Annu. Rev. Plant Physiol. Plant Mol. Biol., 48:89-108). Chemically inducible promoters are particularly suitable when it is desired that the gene expression takes place in a time-specific manner. Examples of such promoters are a salicylic-acid-inducible promoter (WO 95/19443), a tetracycline-inducible promoter (Gatz et al. (1992) Plant J. 2, 397-404) and an ethanol-inducible promoter.

Promoters which respond to biotic or abiotic stress conditions are also suitable, for example the pathogen-induced PRP1 gene promoter (Ward et al., Plant. Mol. Biol. 22 (1993) 361-366), the heat-inducible tomato hsp80 promoter (U.S. Pat. No. 5,187,267), the chill-inducible potato alpha-amylase promoter (WO 96/12814) or the wound-inducible pinII promoter (EP-A-0 375 091).

Especially preferred are those promoters which bring about the gene expression in tissues and organs in which the biosynthesis of fatty acids, lipids and oils takes place, in seed cells, such as cells of the endosperm and of the developing embryo. Suitable promoters are the oilseed rape napin gene promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legume B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley lpt2 or lpt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890.

In particular, it may be desired to bring about the multiparallel expression of the Δ5-desaturase, Δ6-desaturase, Δ12-desaturase, ω3-desaturase or Δ6-elongase used in the process. Such expression cassettes can be introduced via the simultaneous transformation of a plurality of individual expression constructs or, preferably, by combining a plurality of expression cassettes on one construct. Also, a plurality of vectors can be transformed with in each case a plurality of expression cassettes and then transferred into the host cell. For the purpose of the invention, it is also possible to introduce genes into different plants and to combine them by hybridization.

Other preferred sequences for the use in operable linkage in plant gene expression cassettes are targeting sequences which are required for targeting the gene product into its corresponding cell compartment, for example into the vacuole, the nucleus, all types of plastids, such as amyloplasts, chloroplasts, chromoplasts, the extracellular space, the mitochondria, the endoplasmic reticulum, oil bodies, peroxisomes and other compartments of plant cells (for an overview, see Kermode, Crit. Rev. Plant Sci. 15, 4 (1996) 285-423 and references cited therein).

For the purposes of the invention, “transgenic” or “recombinant” means with regard to, for example, a nucleic acid sequence, an expression cassette (=gene construct) or a vector comprising the nucleic acid sequence according to the invention or an organism transformed with the nucleic acid sequences, expression cassettes or vector according to the invention, all those constructions brought about by recombinant methods in which either

a) the nucleic acid sequence according to the invention, or

b) a genetic control sequence which is operably linked with the nucleic acid sequence according to the invention, for example a promoter, or

c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original organism or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences used in the process according to the invention with the corresponding Δ5-desaturases, Δ6-desaturases, Δ12-desaturases, ω3-desaturases, Δ6-elongase—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

Transgenic plants for the purposes of the invention is therefore understood as meaning that the nucleic acids used in the process are not at their natural locus in the genome of the plant, it being possible for the nucleic acids to be expressed homologously or heterologously. However, transgenic also means that, while the nucleic acids according to the invention are at their natural position in the genome of the plant, however, the sequence having been modified with regard to the natural sequence, and/or that the regulatory sequences of the natural sequences have been modified. Transgenic is preferably understood as meaning the expression of the nucleic acids according to the invention or the nucleic acid sequences used in the process according to the invention at an unnatural locus in the genome, i.e. homologous or, preferably, heterologous expression of the nucleic acids takes place. Preferred transgenic plants are oilseed or oil fruit crops, and specifically the different plant parts thereof.

These include plant cells and certain tissues, organs and parts of plants in all their phenotypic forms, such as anthers, fibers, root hairs, stalks, embryos, calli, cotelydons, petioles, harvested material, plant tissue, reproductive tissue and cell cultures, which is derived from the actual transgenic plant and/or can be used for bringing about the transgenic plant.

Transgenic plants or advantageously the seeds thereof which comprise the polyunsaturated fatty acids in particular ARA, EPA and/or their mixtures, synthesized in the process according to the invention can advantageously be marketed directly without there being any need for the oils, lipids or fatty acids synthesized to be isolated. Plants for the process according to the invention are as meaning intact plants and all plant parts, plant organs or plant parts such as lea, stem, seeds, root, tubers, anthers, fibers, root hairs, stalks, embryos, calli, cotelydons, petioles, harvested material, plant tissue, reproductive tissue and cell cultures which are derived from the actual transgenic plant and/or can be used for bringing about the transgenic plant. In this context, the seed comprises all parts of the seed such as the seed coats, epidermal cells, seed cells, endosperm or embryonic tissue.

In principle, the process according to the invention is also suitable for the production of polyunsaturated fatty acids, in particular ARA, EPA and/or their mixtures, in plant cell cultures, followed by obtaining the fatty acids from the cultures. In particular, they may take the form of suspension or callus cultures.

However, the compounds produced in the process according to the invention can also be isolated from the plants, advantageously the plant seeds, in the form of their oils, fat, lipids and/or free fatty acids. Polyunsaturated fatty acids produced by this process can be harvested by harvesting the plants or plant seeds either from the culture in which they grow, or from the field.

In a further preferred embodiment, this process furthermore comprises the step of obtaining the oils, lipids or free fatty acids from the plant or from the crop. The crop may, for example, take the form of a greenhouse- or field-grown plant crop.

The oils, lipids or free fatty acids can be isolated via pressing or extraction of the plant parts, preferably the plant seeds. In this context, the oils, fats, lipids and/or free fatty acids can be obtained by what is known as cold-beating or cold-pressing without applying heat. To allow for greater ease of disruption of the plant parts, specifically the seeds, they are previously commuinuted, steamed or roasted. The seeds which have been pretreated in this manner can subsequently be pressed or extracted with solvents such as warm hexane. The solvent is subsequently removed.

Thereafter, the resulting products which comprise the polyunsaturated fatty acids are processed further, i.e. refined. In this process, substances such as the plant mucilages and suspended matter are first removed. What is known as desliming can be effected enzymatically or, for example, chemico-physically by addition of acid such as phosphoric acid. Thereafter, the free fatty acids are removed by treatment with a base, for example sodium hydroxide solution. The resulting product is washed thoroughly with water to remove the alkali remaining in the product and then dried. To remove the pigment remaining in the product, the products are subjected to bleaching, for example using fuller's earth or active charcoal. At the end, the product is deodorized, for example using steam.

The oils, lipids, fatty acids or fatty acid mixtures according to the invention which are obtained after pressing are referred to as what is known as crude oils. They still comprise all of the oil and/or lipid components and also compounds which are soluble in these. Such compounds are the various tocopherols such as α-tocopherol, β-tocopherol, γ-tocopherol and/or δ-tocopherol or phytosterols such as brassicasterol, campesterol, stigmasterol, β-sitosterol, sitostanol, Δ⁵-avenasterol, Δ⁵,24-stigmastadienol, Δ⁷-stigmasternol or Δ⁷-avenasterol. These compounds are present in a range of from 1 to 1000 mg/100 g, advantageously 10 to 800 mg/100 g of lipid or oil. Triterpenes such as germaniol, amyrin, cycloartenol and others may also be present in these lipids and oils. These lipids and/or oils comprise the polyunsaturated fatty acids produced in the process, such as ARA, EPA and/or DHA, bound in polar and unpolar lipids such as phospholipids, for example phosphatidylcholine, phosphatidylethanolamine, phosphatidylinositol, phosphatidylserine, phosphatidylglycerol, galactolipids, monoglycerides, diglycerides or triglycerides, to mention but a few. Lysophospholipids may also be present in the lipids and/or oils. These components of the lipids and/or oils can be separated from one another by suitable processes. Cholesterol is not present in these crude oils.

A further embodiment according to the invention is the use of the oil, lipid, fatty acids and/or the fatty acid composition in feedstuffs, foodstuffs, cosmetics or pharmaceuticals. The oils, lipids, fatty acids or fatty acid mixtures according to the invention can be used in the manner with which the skilled worker is familiar for mixing with other oils, lipids, fatty acids or fatty acid mixtures of animal origin such as, for example, fish oils. Typical of such fish oils short-chain fatty acids such as C12:0, C14:0, C14:1, branched C15:0, C15:0, C16:0 or C16:1. Polyunsaturated C16-fatty acids such as C16:2, C16:3 or C16:4, branched C17:0, C17:1, branched C18:0 and C19:0 and also C19:0 and C19:1 are also found in fish oil. Such fatty acids are typical of fish oils and are only found rarely, or not at all, in vegetable oils. Economically relevant fish oils are, for example, anchovy oil, menhaden oil, tuna oil, sardine oil, herring oil, mackerel oil, whale oil and salmon oil. These lipids and/or oils of animal origin can be used for mixing with the oils according to the invention in the form of crude oils, i.e. in the form of lipids and/or oils which have not yet been purified, or else various purified fractions may be used for mixing.

The oils, lipids, fatty acids or fatty acid mixtures according to the invention can be used in the manner with which the skilled worker is familiar for mixing with other oils, lipids, fatty acids or fatty acid mixtures of animal origin such as, for example, fish oils. Again, these oils, lipids, fatty acids or fatty acid mixtures, which are composed of vegetable and animal constituents, may be used for the preparation of foodstuffs, feedstuffs, cosmetics or pharmaceuticals.

The term “oil”, “lipid” or “fat” is understood as meaning a fatty acid mixture comprising unsaturated or saturated, preferably esterified, fatty acid(s). The oil, lipid or fat is preferably high in polyunsaturated free or, advantageously, esterified fatty acid(s), in particular linoleic acid, γ-linolenic acid, dihomo-γ-linolenic acid, arachidonic acid, α-linolenic acids stearidonic acid or eicosatetraenoic acid. The amount of unsaturated esterified fatty acids preferably amounts to approximately 30%, a content of 50% is more preferred, a content of 60%, 70%, 80%, 85% or more is even more preferred. For the analysis, the fatty acid content can, for example, be determined by gas chromatography after converting the fatty acids into the methyl esters by transesterification. The oil, lipid or fat can comprise various other saturated or unsaturated fatty acids, for example palmitic acid, palmitoleic acid, stearic acid, oleic acid and the like. The content of the various fatty acids in the oil or fat can vary, in particular depending on the starting organism.

The polyunsaturated fatty acids produced in the process are, as described above, for example sphingolipids, phosphoglycerides, lipids, glycolipids, phospholipids, monoacylglycerol, diacylglycerol, triacylglycerol or other fatty acid esters.

Starting from the lipids, phospholipids or triacylglycerides prepared in the process according to the invention, the polyunsaturated fatty acids which are present can be liberated for example via treatment with alkali, for example aqueous KOH or NaOH, or acid hydrolysis, advantageously in the presence of an alcohol such as methanol or ethanol, or via enzymatic cleavage, and isolated via, for example, phase separation and subsequent acidification via, for example, H₂SO₄. The fatty acids can also be liberated directly without the above-described processing step.

Owing to the process according to the invention, the polyunsaturated fatty acids which have been produced can be increased in the plants used in the process in two ways, in principle. Either the pool of free polyunsaturated fatty acids and/or the content of the esterified polyunsaturated fatty acids produced via the process can be increased. It is advantageous to increase, via the process according to the invention, the pool of esterified polyunsaturated fatty acids in the transgenic organisms.

All the nucleic acid sequences used in the process according to the invention are advantageously derived from a eukaryotic organism such as a plant, a microorganism such as an alga, or an animal. The nucleic acid sequences are preferably derived from the order Salmoniformes, Xenopus or Ciona, algae such as Mantoniella, Crypthecodinium, Euglena or Ostreococcus, fungi such as the genus Phytophthora, or from diatoms such as the genera Thalassiosira or Phaeodactylum.

Nucleic acids which can be used advantageously in the process are derived from bacteria, fungi, diatoms, animals such as Caenorhabditis or Oncorhynchus or plants such as algae or mosses such as the genera Shewanella, Physcomitrella, Thraustochytrium, Fusarium, Phytophthora, Ceratodon, Mantoniella, Ostreococcus, Isochrysis, Aleurita, Muscarioides, Mortierella, Borago, Phaeodactylum, Crypthecodinium, specifically from the genera and species Oncorhynchus mykiss, Xenopus laevis, Ciona intestinalis, Thalassiosira pseudonona, Montoniella squamata, Ostreococcus sp., Ostreococcus tauri, Euglena gracilis, Physcomitrella patens, Phytophthora infestans, Fusarium graminacum, Cryptocodinium cohnii, Ceratodon purpureus, Isochrysis galbana, Aleurita farinosa, Thraustochytrium sp., Muscarioides viallii, Mortierella alpina, Borago officinalis, Phaeodactylum tricornutum, Caenorhabditis elegans or especially advantageously from Oncorhynchus mykiss, Euglena gracilis, Thalassiosira pseudonana or Crypthecodinium cohnii.

This invention is illustrated in greater detail by the examples which follow, which are not to be construed as limiting. The content of all of the references, patent applications, patents and published patent applications cited in the present patent application is herewith incorporated by reference.

### EXAMPLES

### Example 1

### General Cloning Methods

The cloning methods such as, for example, restriction cleavages, agarose gel electrophoresis, purification of DNA fragments, transfer of nucleic acids to nitrocellulose and nylon membranes, linkage of DNA fragments, transformation of E. coli cells, bacterial cultures and the sequence analysis of recombinant DNA were carried out as described by Sambrook et al. (1989) (Cold Spring Harbor Laboratory Press: ISBN 0-87969-309-6).

### Example 2

### Sequence Analysis of Recombinant DNA

Recombinant DNA molecules were sequenced with an ABI laser fluorescence DNA sequencer by the process of Sanger (Sanger et al. (1977) Proc. Natl. Acad. Sci. USΔ74, 5463-5467). Fragments resulting from a polymerase chain reaction were sequenced and verified to avoid polymerase errors in constructs to be expressed.

### Example 3

### Cloning Expression Plasmids for the Seed-Specific Expression in Plants

Unless otherwise described, the general conditions described hereinbelow apply to all subsequent experiments.

The following are used by preference in accordance with the invention for the examples which follow: Bin19, pBI101, pBinAR, pGPTV and pCAMBIA. An overview over binary vectors and their use can be found in Hellens et al., Trends in Plant Science (2000) 5, 446-451. A pGPTV derivative as described in DE 10205607 was used. This vector differs from pGPTV by an AscI restriction cleavage site which had additionally been introduced.

The starting point of the cloning procedure was the cloning vector pUC 19 (Maniatis et al.). In the first step, the conlinin promoter fragment was amplified, using the following primers:

| | |
|---|---|
| Cnl1 C 5′: | gaattcggcgcgccgagctcctcgagcaacggttccggcggtatagagttgggtaattcga |
| Cnl1 C 3′: | cccgggatcgatgccggcagatctccaccattttttggtggtgat |

Composition of the PCR mix (50 μl):

5.00 μl template cDNA

5.00 μl 10× buffer (Advantage polymerase)+25 mM MgCl₂

5.00 μl of 2 mM DNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme EcoRI and then for 12 hours at 25° C. with the restriction enzyme SmaI. The cloning vector pUC19 was incubated in the same manner. Thereafter, the PCR product and the 2668 bp, cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1-C was verified by sequencing.

In the next step, the OCS terminator (Genbank Accession V00088; De Greve, H., Dhaese, P., Seurinck, J., Lemmers, M., Van Montagu, M. and Schell, J. Nucleotide sequence and transcript map of the Agrobacterium tumefaciens Ti plasmid-encoded octopine synthase gene J. Mol. Appl. Genet. 1 (6), 499-511 (1982)) from the vector pGPVT-USP/OCS (DE 102 05 607) was amplified using the following primers:

| | |
|---|---|
| OCS_C 5′: | aggcctccatggcctgctttaatgagatatgcgagacgcc |
| OCS_C 3′: | cccgggccggacaatcagtaaattgaacggag |

Composition of the PCR mix (50 μl):

5.00 μl template cDNA

5.00 μl 10× buffer (Advantage polymerase)+25 mM MgCl₂

5.00 μl of 2 mM DNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme StuI and then for 12 hours at 25° C. with the restriction enzyme SmaI. The vector pUC19-Cnl1-C was incubated for 12 hours at 25° C. with the restriction enzyme SmaI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1-C_OCS was verified by sequencing.

In the next step, the Cnl1-B promoter was amplified by means of PCR, using the following primers:

| | |
|---|---|
| Cnl1-B 5′: | aggcctcaacggttccggcggtatag |
| Cnl1-B 3′: | cccggggttaacgctagcgggcccgatatcggatcccattttttggtggtgattggttct |

Composition of the PCR mix (50 μl):

5.00 μl template cDNA

5.00 μl 10× buffer (Advantage polymerase)+25mM MgCl₂

5.00 μl of 2 mM dNTP

1.25 μl of each primer (10 pmol/μl)

0.50 μl of Advantage polymerase (Clontech).

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme StuI and then for 12 hours at 25° C. with the restriction enzyme SmaI. The vector pUC19-Cnl1-C was incubated for 12 hours at 25° C. with the restriction enzyme SmaI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1-C_Cnl1B_OCS was verified by sequencing.

In a further step, the OCS terminator for Cnl1B was inserted To this end, the PCR was carried out with the following primers:

| | |
|---|---|
| OCS2 5′: | aggcctcctgctttaatgagatatgcgagac |
| OCS2 3′: | cccgggcggacaatcagtaaattgaacggag |

Composition of the PCR mix (50 μl):

5.00 μl template cDNA

5.00 μl 10× buffer (Advantage polymerase)+25 mM MgCl₂

5.00 μl of 2 mM dNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme StuI and then for 12 hours at 25° C. with the restriction enzyme SmaI. The vector pUC19-Cnl1-C_Cnl1B_OCS was incubated for 12 hours at 25° C. with the restriction enzyme SmaI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1-C_Cnl1B_OCS2 was verified by sequencing.

In the next step, the Cnl1-A promoter was amplified by means of PCR, using the following primers:

Cnl1-B 5′:
aggcctcaacggttccggcggtatagag
Cnl1-B 3′:
aggccttctagactgcaggcggccgcccgcattttttggtggtgattggt

Composition of the PCR mix (50 μl):

5.00 μl template cDNA

5.00 μl 10× buffer (Advantage polymerase)+25mM MgCl₂

5.00 μl of 2 mM DNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme StuI. The vector pUCl19-Cnl1-C was incubated for 12 hours at 25° C. with the restriction enzyme SmaI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1C_Cnl1-B_Cnl1A_OCS2 was verified by sequencing.

In a further step, the OCS terminator for Cnl1A was inserted. To this end, the PCR was carried out with the following primers:

OCS2 5′:
ggcctcctgctttaatgagatatgcga
OCS2 3′:
aagcttggcgcgccgagctcgtcgacggacaatcagtaaattgaacggag
a

Composition of the PCR mix (50 μl):

5.00 μl template cDNA

5.00 μl 10× buffer (Advantage polymerase)+25 mM MgCl₂

5.00 μl of 2 mM dNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme StuI and then for 12 hours at 37° C. with the restriction enzyme HindIII. The vector pUC19-Cnl1C_Cnl1-B_Cnl1A_OCS2 was incubated for 2 hours at 37° C. with the restriction enzyme StuI and for 2 hours at 37° C. with the restriction enzyme HindIII. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1C_Cnl1-B_Cnl1A_OCS3 was verified by sequencing.

In the next step, the plasmid pUC19-Cnl1C_Cnl1-B_CNl1A_OCS3 was used for cloning the Δ6-, Δ5-desaturase and the Δ6-elongase. To this end, the Δ6-desaturase from Phytium irregulare (WO02/26946) was amplified using the following PCR primers:

| | |
|---|---|
| D6Des(Pir) 5′: | agatctatggtggacctcaagcctggagtg |
| D6Des(Pir) 3′: | ccatggcccgggttacatcgttgggaactcggtgat |

Composition of the PCR mix (50 μl):

5.00 μl template cDNA

5.00 μl 10× buffer (Advantage polymerase)+25mM MgCl₂

5.00 μl of 2 mM dNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme BglII and then for 2 hours at 37° C. with the restriction enzyme NcoI. The vector pUC19-Cnl1C_Cnl1-B_Cnl1A_OCS3 was incubated for 2 hours at 37° C. with the restriction enzyme BglII and for 2 hours at 37° C. with the restriction enzyme NcoI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1_d6Des(Pir) was verified by sequencing.

In the next step, the plasmid pUC19-Cnl1_d6Des(Pir) was used for cloning the Δ5-desaturase from Thraustochytrium ssp. (WO02/26946). To this end, the Δ5-desaturase from Thraustochytrium ssp. was amplified using the following PCR primers:

| | |
|---|---|
| D5Des(Tc) 5′: | gggatccatgggcaagggcagcgagggccg |
| D5Des(Tc) 3′: | ggcgccgacaccaagaagcaggactgagatatc |

Composition of the PCR mix (50 μl):

5.00 μl template EDNA

5.00 μl 10× buffer (Advantage polymerase)+25 mM MgCl₂

5.00 μl of 2 mM DNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme BamHI and then for 2 hours at 37° C. with the restriction enzyme EcoRV. The vector pUC19-Cnl1_d6Des(Pir) was incubated for 2 hours at 37° C. with the restriction enzyme BamHI and then for 2 hours at 37° C. with the restriction enzyme EcoRV. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1_d6Des(Pir) d5Des(Tc) was verified by sequencing.

In the next step, the plasmid pUC19-Cnl1_d6Des(Pir)-dsDes(Tc) was used for cloning the Δ6-elongase from Physcomityella patens (WO01/59128), to which end the latter was amplified with the following PCR primers:

| | |
|---|---|
| D6Elo(Pp) 5′: | gcggccgcatggaggtcgtggagagattctacggtg |
| D6Elo(Pp) 3′: | gcaaaagggagctaaaactgagtgatctaga |

Composition of the PCR mix (50 μl):

5.00 μl template cDNA

5.00 μl 10× buffer (Advantage polymerase)+25 mM MgCl₂

5.00 μl of 2 mM dNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme NotI and then for 2 hours at 37° C. with the restriction enzyme XbaI. The vector pUC19-Cnl1_d6Des(Pir)_d5Des(Tc) was incubated for 2 hours at 37° C. with the restriction enzyme NotI and for 2 hours at 37° C. with the restriction enzyme XbaI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1_d6Des(Pir)_d5Des(Tc)_D6Elo(Pp) was verified by sequencing.

Starting from pUC19-Cnl1_d6Des(Pir)_d5Des(Tc)_D6Elo(Pp), the binary vector for the plant transformation was prepared. To this end, pUC19-Cnl1_d6Des(Pir)_d5Des(Tc)_D6Elo(Pp) was incubated for 2 hours at 37° C. with the restriction enzyme AscI. The vector pGPTV was treated in the same manner. Thereafter, the fragment from pUC19-Cnl1_d6Des(Pir)_d5Des(Tc)_D6Elo(Pp) and the cleaved pGPTV vector were separated by agarose gel electrophoresis, and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pGPTV-Cnl1_d6Des(Pir)_d5Des(Tc)_D6Elo(Pp) was verified by sequencing.

A further construct, pGPTV-Cnl1_d6Des(Pir)_d5Des(Tc)_D6Elo(Pp)_D12Des(Co), was used. To this end, an amplification was carried out with the following primers, starting from pUC19-Cnl1C_OCS:

| | |
|---|---|
| Cnl1_OCS 5′: | gtcgatcaacggttccggcggtatagagttg |
| Cnl1_OCS 3′: | gtcgatcggacaatcagtaaattgaacggaga |

Composition of the PCR mix (50 μl):

5.00 μl template cDNA

5.00 μl 10× buffer (Advantage polymerase)+25 mM MgCl₂

5.00 μl of 2 mM dNTP

1.25 μl of each primer (10 pmol/μl)

0.50 μl of Advantage polymerase (Clontech)

PCR reaction conditions:

Annealing temperature. 1 min 55° C.

Denaturation temperature: 1 min 94° C.

Elongation temperature: 2 min 72° C.

Number of cycles: 35

The PCK product was first incubated for 2 hours at 37° C. with the restriction enzyme SalI. The vector pUC19 was incubated for 2 hours at 37° C. with the restriction enzyme SalI. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1_OCS was verified by sequencing.

In a further step, the Δ12-desaturase gene from Calendula officinalis (WO01/85968) was cloned into pUC19-Cnl1_OCS. To this end, d12Des(Co) was amplified with the following primers:

| | |
|---|---|
| D12Des(Co) 5′: | agatctatgggtgcaggcggtcgaatgc |
| D12Des(Co) 3′; | ccatggttaaatcttattacgatacc |

Composition of the PCR mix (50 μl):

5.00 μl template cDNA

5.00 μl 10× buffer (Advantage polymerase)+25 mM MgCl₂

5.00 μl of 2 mM dNTP

1.25 μl of each primer (10 pmol/μl)

0.50 μl of Advantage polymerase (Clontech)

PCR reaction conditions:

Annealing temperature: 1 min 55° C.

Denaturation temperature: 1 min 94° C.

Elongation temperature: 2 min 72° C.

Number of cycles: 35

The PCR product was first incubated for 2 hours at 37° C. with the restriction enzyme BglII and then for 2 hours at the same temperature with NcoI. The vector pUC19-Cnl1_OCS was incubated in the same way. Thereafter, the PCR product and the cleaved vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pUC19-Cnl1_D12Des(Co) was verified by sequencing.

Plasmid pUC19-Cnl1_D12Des(Co) and plasmid pUC19-Cnl1_{—d}6Des(Pir)_d5Des(Tc)_D6Elo(Pp) were incubated for 2 hours at 37° C. with the restriction enzyme SalI. Thereafter, the vector fragment and the cleaved vector were separated by agarose gel electrophoresis and the corresponding DNA fragments were excised. The DNA was purified by means of the Qiagen Gel Purification Kit following the manufacturer's instructions. Thereafter, the vector and the vector fragments were ligated. The Rapid Ligation Kit from Roche was used for this purpose. The resulting plasmid pUC19-Cnl1_d6Des(Pir)_d5Des(Tc)_D6Elo(Pp)_D12Des(Co) was verified by sequencing.

The binary vector for the transformation of plants was prepared starting from pUC19-Cnl1_d6Des(Pir)_d5Des(Tc)_D6Elo(Pp)_D12Des(Co). To this end, pUC19-Cnl1_d6Des(Pir)_d5Des(Tc)_D6Elo(Pp)_D12Des(Co) was incubated for 2 hours at 37° C. with the restriction enzyme AscI. The vector pGPTV was treated in the same manner. Thereafter, the fragment from pUC19-Cnl1_d6Des(Pir)_d5Des(Tc)D6Elo(Pp)_D12Des(Co) and the cleaved pGPTV vector were separated by agarose gel electrophoresis and the corresponding DNA fragments were excised. The DNA was purified by means of the Qiagen Gel Purification Kit following the manufacturer's instructions. Thereafter, the vector and the PCR product were ligated. The Rapid Ligation Kit from Roche was used for this purpose. The resulting plasmid pGPTV-Cnl1_d6Des(Pir)_d5Des(Tc)_D6Elo(Pp)_D12Des(Co) was verified by sequencing.

A further vector which is suitable for the transformation of plants is pSUN2. In order to increase the number of expression cassettes present in the vector to more than four, this vector was used in combination with the Gateway system (Invitrogen, Karlsruhe). To this end, the Gateway cassette A was introduced into the vector pSUN2 in accordance with the manufacturer's instructions, as described hereinbelow:

The pSUN2 vector (1 μg) was incubated for 1 hour with the restriction enzyme EcoRV at 37° C. Thereafter, the Gateway cassette A (Invitrogen, Karlsruhe) was ligated into the cleaved vector by means of the Rapid Ligation Kit from Roche, Mannheim. The resulting plasmid was transformed into E. coli DB3.1 cells (Invitrogen). The insulated plasmid pSUN-GW was subsequently verified by sequencing.

In the second step, the expression cassette was excised from pUC19-Cnl1_d6Des(Pir)_d5Des(Tc)_D6Elo(Pp)_D12Des(Co) by means of AscI and ligated into the vector pSUN-GW, which had been treated in the same manner.

### Example 4

### Generation of Transgenic Plants

a) Generation of transgenic Brassica plants (modified according to the process of Moloney et al., 1992, Plant Cell Reports, 8:238-242)

The binary vectors in Agrobacterium tumefaciens C58C1:pGV2260 or Escherichia coli (Deblaere et al, 1984, Nucl. Acids. Res. 13, 4777-4788) can be used for generating transgenic oilseed rape plants. To transform oilseed rape plants (Var. Drakkar, NPZ Nordeutsche Pflanzenzucht, Hohenlieth, Germany), a 1:50 dilution of an overnight culture of a positively transformed agrobacterial colony in Murashige-Skoog medium (Murashige and Skoog 1962 Physiol. Plant. 15, 473) supplemented with 3% sucrose (3MS medium) is used. Petioles or hypocotyls of freshly germinated sterile oilseed rape plants (in each case approx. 1 cm²) are incubated with a 1:50 agrobacterial dilution for 5-10 minutes in a petri dish. This is followed by 3 days of coincubation in the dark at 25° C. on 3MS medium supplemented with 0.8% Bacto agar. The cultures are then grown for 3 days at 16 hours light/8 hours dark. The cultivation is then continued in a weekly rhythm on MS medium supplemented with 500 mg/l Claforan (cefotaxime sodium), 50 mg/l kanamycin, 20 μM benzylaminopurine (BAP) and 1.6 g/l of glucose. Growing shoots are transferred to MS medium supplemented with 2% sucrose, 250 mg/l Claforan and 0.8% Bacto agar. If no roots have developed after three weeks, 2-indolebutyric acid is added to the medium as growth hormone for rooting.

Regenerated shoots were obtained on 2MS medium supplemented with kanamycin and Claforan; after rooting, they were transferred to compost and, after growing on for two weeks in a controlled-environment cabinet or in the greenhouse, allowed to flower, and mature seeds were harvested and analyzed by lipid analysis for elongase expression such as Δ6-elongase activity or ω3-desaturase activity. In this manner, lines with elevated contents of polyunsaturated C₂₀- and C₂₂-fatty acids can be identified.

b) Generation of transgenic Camelina plants

Agrobacterium tumefaciens strain C58 was transformed with the PUFA vector 81, 191 and 192 by means of electroporation. Explants of Camelina seedlings (age >1 week), which had been grown on MS medium, were inoculated with agrobacteria. After two weeks of coculture, the plants were washed in order to remove the agrobacteria and subsequently transferred to regeneration medium with optimized BaP and NAA. After a further two days' regeneration, optimized amounts of kanamycin were added. This selection pressure was maintained for 12 days. Shoot regeneration was initiated by transfer onto kanamycin-free medium comprising BaP. Shoot formation was complete after >3 weeks post-inoculation, and root formation was induced on medium comprising NAA. After rooting, shoots were transferred into compost and grown in a controlled-environment cabinet or in the greenhouse, they were allowed to flower, and mature seeds were harvested and tested for elongase expression such as Δ6-elongase activity or ω3-desaturase activity by means of lipid analysis. In this manner, lines with an increased content of polyunsaturated fatty acids were identified.

### Example 5

### Lipid Extraction from Seeds

The effect of the genetic modification in plants on the production of a desired compound (such as a fatty acid) can be determined by growing the modified plant under suitable conditions (such as those described above) and analyzing the medium and/or the cellular components for the elevated production of the desired product (i.e. of the lipids or a fatty acid). These analytical techniques are known to the skilled worker and comprise spectroscopy, thin-layer chromatography, various types of staining methods, enzymatic and microbiological methods and analytical chromatography such as high-performance liquid chromatography (see, for example, Ullman, Encyclopedia of Industrial Chemistry, Vol. A2, p. 89-90 and p. 443-613, VCH: Weinheim (1985); Fallon, A., et al., (1987) “Applications of HPLC in Biochemistry” ins Laboratory Techniques in Biochemistry and Molecular Biology, Vol. 17; Rehm et al. (1993) Biotechnology, Vol. 3, Chapter III: “Product recovery and purification”, p. 469-714, VCH: Weinheim; Belter, P. A., et al. (1988) Bioseparations: downstream processing for Biotechnology, John Wiley and Sons; Kennedy, J. F., and Cabral, J. M. S. (1992) Recovery processes for biological Materials, John Wiley and Sons; Shaeiwitz, J. A., and Henry, J. D. (1988) Biochemical Separations, in: Ullmann's Encyclopedia of Industrial Chemistry, Vol. B3; Chapter 11, p. 1-27, VCH: Weinheim; and Dechow, F. J. (1989) Separation and purification techniques in biotechnology, Noyes Publications).

In addition to the abovementioned methods, plant lipids are extracted from plant material as described by Cahoon et al. (1999) Proc. Natl. Acad. Sci. USA 96 (22);12935-12940 and Browse et al. (1986) Analytic Biochemistry 152:141-145.

The qualitative and quantitative analysis of lipids or fatty acids is described by Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 pp. (Oily Press Lipid Library; 1); “Progress in Lipid Research, Oxford: Pergamon Press, 1 (952)-16 (1977) under the title: Progress in the Chemistry of Fats and Other Lipids CODEN.

In addition to measuring the end product of the fermentation, it is also possible to analyze other components of the metabolic pathways which are used for the production of the desired compound, such as intermediates and by-products, in order to determine the overall production efficiency of the compound. The analytical methods comprise measuring the amount of nutrients in the medium (for example sugars, hydrocarbons, nitrogen sources, phosphate and other ions), measuring the biomass composition and the growth, analyzing the production of conventional metabolites of biosynthetic pathways and measuring gases which are generated during the fermentation. Standard methods for these measurements are described in Applied Microbial Physiology; A Practical Approach, P. M. Rhodes and P. F. Stanbury, Ed., IRL Press, p. 103-129; 131-163 and 165-192 (ISBN: 0199635773) and references cited therein.

One example is the analysis of fatty acids (abbreviations: FAME, fatty acid methyl ester; GC-MS, gas liquid chromatography/mass spectrometry; TAG, triacylglycerol; TLC, thin-layer chromatography).

The unambiguous detection for the presence of fatty acid products can be obtained by analyzing recombinant organisms using analytical standard methods: GC, GC-MS or TLC, as described on several occasions by Christie and the references therein (1997, in: Advances on Lipid Methodology, Fourth Edition: Christie, Oily Press, Dundee, 119-169; 1998, Gaschromatographie-Massenspektrometrie-Verfahren [Gas chromatography/mass spectrometric methods], Lipide 33:343-353).

The material to be analyzed can be disrupted by sonication, grinding in a glass mill, liquid nitrogen and grinding or via other applicable methods. After disruption, the material must be centrifuged. The sediment is resuspended in distilled water, heated for 10 minutes at 100° C., cooled on ice and recentrifuged, followed by extraction for one hour at 90° C. in 0.5 M sulfuric acid in methanol with 2% dimethoxypropane, which leads to hydrolyzed oil and lipid compounds, which give transmethylated lipids. These fatty acid methyl esters are extracted in petroleum ether and finally subjected to a GC analysis using a capillary column (Chrompack, WCOT Fused Silica, CP-Wax-52 CB, 25 m, 0.32 mm) at a temperature gradient of between 170° C. and 240° C. for 20 minutes and 5 minutes at 240° C. The identity of the resulting fatty acid methyl esters must be defined using standards which are available from commercial sources (i.e. Sigma).

Plant material is initially homogenized mechanically by comminuting in a pestle and mortar to make it more amenable to extraction.

This is followed by heating at 100° C. for 10 minutes and, after cooling on ice, by resedimentation. The cell sediment is hydrolyzed for one hour at 90° C. with 1 M methanolic sulfuric acid and 2% dimethoxypropane, and the lipids are transmethylated. The resulting fatty acid methyl esters (FAMEs) are extracted in petroleum ether. The extracted FAMEs are analyzed by gas liquid chromatography using a capillary column (Chrompack, WCOT Fused Silica, CP-Wax-52 CB, 25 m, 0.32 mm) and a temperature gradient of from 170° C. to 240° C. in 20 minutes and 5 minutes at 240° C. The identity of the fatty acid methyl esters is confirmed by comparison with corresponding FAME standards (Sigma). The identity and position of the double bond can be analyzed further by suitable chemical derivatization of the FAME mixtures, for example to give 4,4-dimethoxyoxazolin derivatives (Christie, 1998) by means of GC-MS.

### Example 6

### Analysis of the Seeds From the Transgenic Plants Which Have Been Generated

Analogously to Example 5, the seeds of the plants which had been transformed with the constructs pGPTV-Cnl1_d6Des(Pir)_d5Des(Tc)_D6Elo(Pp)_D12Des(Co), pSUN-SG and pSUN-8G were analyzed. In comparison with control plants which were not transformed (wild-type control, WT), a pronounced change in the fatty acid spectrum was observed. It was thus possible to demonstrate that the transformed genes are functional. Table 1 compiles the results.

TABLE 1

| | Fatty acids | | | | | | | | |
|---|---|---|---|---|---|---|---|---|---|
| Lines | 16:0 | 18:0 | 18:1 | 18:2 | GLA | 18:3 | SDA | ARA | EPA |
| WT control | 5.6 | 6.5 | 31.7 | 41.7 | nd | 12.1 | nd | nd | nd |
| 1424_Ko82_4 | 6.6 | 1.5 | 8.9 | 10.5 | 42.2 | 3.1 | 2.8 | 17.2 | 0.2 |
| 1424_Ko82_5 | 6.1 | 1.5 | 11.0 | 9.0 | 40.6 | 2.9 | 4.0 | 15.0 | 1.5 |
| 1424_Ko82_6 | 5.7 | 1.6 | 15.5 | 10.6 | 37.1 | 3.0 | 3.2 | 14.6 | 0.2 |
| 1424_Ko82_7 | 5.4 | 2.0 | 20.4 | 10.7 | 32.6 | 3.5 | 3.2 | 12.1 | 1.0 |
| 1424_Ko82_8 | 5.4 | 1.4 | 15.1 | 12.5 | 39.9 | 2.6 | 2.4 | 12.2 | 0.7 |
| 1424_Ko82_9 | 6.0 | 1.8 | 25.0 | 9.9 | 29.7 | 2.2 | 2.5 | 10.2 | 0.8 |
| 1424_Ko82_10 | 5.7 | 1.3 | 10.1 | 10.3 | 42.5 | 2.6 | 3.5 | 13.9 | 1.1 |
| 1424_Ko82_11 | 5.4 | 1.4 | 15.7 | 11.3 | 38.2 | 2.6 | 2.8 | 14.1 | 1.0 |

Here, the analysis of the seeds with the construct pSUTN-5G reveals lines with a pronounced increase in the arachidonic acid content in comparison with the construct pGPTV-Cnl1_d6Des(Pir)_d5Des(Tc)_D6Elo(Pp)_D12Des(Co). In this context, lines with up to 25% ARA were obtained. The results from this line are compiled in Table 2.

TABLE 2
Fatty acid analysis of transgenic seeds which have been transformed
with the construct pSUN-5G.

| | Fatty acids | | | | | | | | | |
|---|---|---|---|---|---|---|---|---|---|---|
| | | | | | | 18:3 | 18:4 | 20:3 | | |
| Lines | 16:0 | 18:0 | 18:1 | 18:2 LA | 18:3 GLA | ALA | SDA | HGLA | ARA | EPA |
| WT | 5.2 | 2.3 | 34.2 | 37.9 | 0.0 | 11.6 | 0.0 | 0.0 | 0.0 | 0.0 |
| 16-1-2 | 4.2 | 1.6 | 20.1 | 21.5 | 25.9 | 4.1 | 1.8 | 1.7 | 8.9 | 0.8 |
| 16-1-3 | 5.8 | 2.3 | 9.9 | 14.6 | 33.6 | 3.1 | 2.2 | 2.2 | 16.0 | 1.4 |
| 16-1-8 | 5.0 | 2.8 | 11.1 | 12.6 | 34.9 | 2.2 | 1.8 | 2.6 | 16.3 | 1.2 |
| 16-2-1 | 4.9 | 1.6 | 14.5 | 17.4 | 32.9 | 3.5 | 2.0 | 1.6 | 12.3 | 1.0 |
| 16-2-5 | 5.5 | 3.3 | 12.9 | 13.8 | 32.9 | 2.9 | 2.2 | 1.4 | 15.4 | 1.4 |
| 16-4-2 | 5.8 | 2.5 | 18.8 | 14.7 | 32.0 | 3.5 | 2.3 | 1.2 | 12.0 | 1.2 |
| 16-4-3 | 5.9 | 2.0 | 19.7 | 15.0 | 32.0 | 3.8 | 2.4 | 1.1 | 11.4 | 1.2 |
| 16-7-2 | 6.2 | 4.4 | 14.3 | 10.2 | 30.7 | 2.0 | 2.1 | 1.7 | 19.4 | 1.9 |
| 16-7-3 | 5.0 | 2.5 | 21.6 | 13.6 | 30.7 | 2.1 | 1.8 | 1.5 | 12.6 | 1.1 |
| 16-7-4 | 5.3 | 4.1 | 18.8 | 19.5 | 23.1 | 4.2 | 2.2 | 2.9 | 11.3 | 1.4 |
| 16-7-5 | 7.4 | 1.8 | 4.2 | 6.8 | 33.7 | 1.8 | 2.7 | 2.6 | 25.8 | 2.6 |

### Example 7

### Analysis of Transgenic Seed Material of Camelina saliva L.

The extraction of seeds of transgenic Camelina sativa plants transformed with PUFA (comprising: Δ6Des(Pir) SEQ ID NO: 1_{—}Δ5Des(Tc) SEQ ID NO: 3_{—}Δ6Elo(Pp) SEQ ID NO: 5_{—}Δ12Des(Co) SEQ ID NO: 11) and the gas-chromatographic analysis were carried out as described in Example 5. Table 3 shows the results of the analyses. The various fatty acids are shown as percent area. It was possible to demonstrate for the first time the synthesis of long-chain polyunsaturated fatty acids in Camelina sativa. Surprisingly, the content of erucic acid (22:1) and icosenoic acid (20:1) was markedly reduced by introducing the synthesis pathway for the production of long-chain polyunsaturated fatty acids, although nothing had been changed in the direct synthesis pathway for erucic acid as described by Mietkiewska, et al., Plant Phys 2004 or Katavic et al., 2002 Europ. Journal of Biochem.

TABLE 3
Gas-chromatographic analysis of seed material from Camelina sativa
I. The individual fatty acids are indicated as percent area.

| | 16:0 | 18:0 | 18:1Δ9 | 18:1Δ11 | 18:2 | γ18:3 | α18:3 |
|---|---|---|---|---|---|---|---|
| PUFA81_1 | 9.4 | 4.6 | 3.5 | 1.1 | 8.0 | 26.6 | 9.5 |
| PUFA81_2 | 12.7 | 4.4 | 4.8 | 0.9 | 19.3 | 15.9 | 18.0 |
| PUFA81_3 | 13.1 | 4.7 | 4.2 | 1.2 | 17.1 | 18.1 | 13.1 |
| PUFA81_4 | 9.2 | 3.2 | 3.7 | 1.1 | 5.8 | 23.7 | 10.8 |
| PUFA81_6 | 8.1 | 4.8 | 1.1 | 0.0 | 9.3 | 19.9 | 14.6 |
| PUFA81_7 | 8.0 | 3.3 | 4.3 | 0.9 | 7.8 | 21.1 | 13.7 |
| PUFA81_8 | 7.9 | 3.7 | 5.0 | 1.2 | 8.5 | 19.9 | 16.1 |
| PUFA81_9 | 8.0 | 3.5 | 4.6 | 1.1 | 7.3 | 21.9 | 13.1 |
| PUFA81_10 | 8.2 | 3.9 | 7.2 | 1.5 | 9.1 | 20.4 | 15.2 |
| PUFA81_16 | 8.0 | 3.4 | 4.7 | 0.8 | 8.4 | 22.0 | 14.8 |
| PUFA81_20 | 8.7 | 3.2 | 5.9 | 1.4 | 8.1 | 21.8 | 14.3 |
| wt_1 | 5.7 | 2.3 | 12.6 | 0.7 | 12.1 | n.n. | 41.6 |
| wt_2 | 6.6 | 1.9 | 10.1 | 0.9 | 14.3 | n.n. | 42.4 |
| wt_3 | 5.6 | 1.9 | 8.9 | 0.8 | 15.0 | n.n. | 43.1 |
| wt_4 | 6.1 | 2.0 | 9.4 | 0.9 | 15.3 | n.n. | 42.9 |
| wt_5 | 6.3 | 2.2 | 10.6 | 0.9 | 15.9 | n.n. | 41.3 |
| wt_6 | 6.2 | 2.4 | 7.5 | 0.8 | 14.8 | n.n. | 43.2 |

| | | | | | 20:4 | | |
|---|---|---|---|---|---|---|---|
| | | | | 20:3 | (ARA) | 20:5 (EPA) | |
| | 18:4 | 20:0 | 20:1 | (11, 14, 17) | (5, 8, 11, 14) | (5, 8, 11, 14, 17) | 22:1 |
| PUFA81_1 | 10.6 | 2.5 | 7.4 | 1.0 | 9.2 | 3.3 | 1.6 |
| PUFA81_2 | 5.3 | 2.9 | 0.5 | 0.7 | 6.7 | 1.8 | 3.1 |
| PUFA81_3 | 8.9 | 2.7 | 0.4 | 0.6 | 7.9 | 3.4 | 1.9 |
| PUFA81_4 | 12.1 | 2.0 | 9.8 | 1.4 | 8.3 | 3.3 | 2.8 |
| PUFA81_6 | 11.7 | 3.6 | 10.1 | 1.5 | 6.3 | 3.0 | 3.1 |
| PUFA81_7 | 11.1 | 2.7 | 9.1 | 1.4 | 7.6 | 3.3 | 3.0 |
| PUFA81_8 | 11.8 | 2.6 | 9.5 | 1.7 | 6.3 | 3.1 | 0.1 |
| PUFA81_9 | 11.3 | 2.4 | 7.7 | 1.4 | 8.8 | 3.7 | 2.3 |
| PUFA81_10 | 10.7 | 2.1 | 8.0 | 1.1 | 6.0 | 2.7 | 1.7 |
| PUFA81_16 | 8.4 | 1.8 | 12.8 | 1.6 | 6.1 | 2.1 | 2.4 |
| PUFA81_20 | 10.1 | 1.5 | 10.3 | 1.3 | 6.3 | 2.7 | 2.1 |
| wt_1 | n.n. | 1.4 | 14.2 | 1.7 | n.n. | n.n. | 3.7 |
| wt_2 | n.n. | 1.2 | 13.1 | 2.1 | n.n. | n.n. | 3.2 |
| wt_3 | n.n. | 1.4 | 12.9 | 2.0 | n.n. | n.n. | 4.0 |
| wt_4 | n.n. | 1.3 | 12.3 | 1.9 | n.n. | n.n. | 3.4 |
| wt_5 | n.n. | 1.5 | 12.5 | 1.7 | n.n. | n.n. | 3.3 |
| wt_6 | n.n. | 1.9 | 12.3 | 1.9 | n.n. | n.n. | 3.9 |

Equivalents:

Many equivalents of the specific embodiments according to the invention described herein can be seen or found by the skilled worker by simple routine experimentation. These equivalents are intended to be comprised by the patent claims.

1.-15. (canceled)

16. A process for the production of arachidonic acid (=ARA) or eicosapentaenoic acid (=EPA) or arachidonic acid and eicosapentaenoic acid in transgenic plants of the Brassicaceae family with an ARA or EPA or ARA and EPA content of at least 3% by weight based on the total lipid content of the transgenic plant, comprising
a) introducing, into a useful plant, at least one nucleic acid sequence which encodes a Δ6-desaturase,
b) introducing, into the useful plant, at least one nucleic acid sequence which encodes a Δ6-elongase,
c) introducing, into the useful plant, at least one nucleic acid sequence which encodes a Δ5-desaturase, and
d) harvesting the useful plant,
where, as the result of the enzymatic activity of the enzymes introduced in steps a) to c), a fatty acid selected from the group consisting of the fatty acids oleic acid [C18:1^{Δ9}], linoleic acid [C18:2^{Δ9, 12}], α-linolenic acid [C18:3^{Δ6, 9, 12}], eicosenoic acid [20:1^{Δ11}] and erucic acid [C22:1^{Δ11}] is reduced by at least 10%, in comparison with a nontransgenic wild-type plant.

17. The process of claim 16, wherein the nucleic acid sequence which encodes a polypeptide with Δ6-desaturase, Δ6-elongase, or Δ5-desaturase activity comprises a nucleic acid sequence selected from the group consisting of:
a) a nucleic acid sequence comprising the sequence shown in SEQ ID NO: 1, SEQ ID NO: 3, SEQ ID NO: 5 or SEQ ID NO: 7;
b) a nucleic acid sequence comprising a nucleic acid sequence encoding a polypeptide comprising the amino acid sequence shown in SEQ ID NO: 2, SEQ ID NO: 4, SEQ ID NO: 6, or SEQ ID NO: 8; and
c) a nucleic acid sequence encoding a polypeptide comprising an amino acid sequence having at least 40% identity at the amino acid level with SEQ ID NO: 2, SEQ ID NO: 4, SEQ ID NO: 6, or SEQ ID NO: 8 and which has a Δ6-desaturase, Δ6-elongase, or Δ5-desaturase activity.

18. The process of claim 16, wherein a nucleic acid sequence which encodes an ω3-desaturase or a Δ12-desaturase or an ω3-desaturase and a Δ12-desaturase is additionally introduced into the useful plants.

19. The process of claim 18, wherein the nucleic acid sequence which encodes a polypeptide with a ω3-desaturase comprises a nucleic acid sequence selected from the group consisting of:
a) a nucleic acid sequence with the sequence shown in SEQ ID NO: 9;
b) a nucleic acid sequence encoding a polypeptide comprising the amino acid sequence shown in SEQ ID NO: 10; and
c) a nucleic acid sequence encoding a polypeptide comprising an amino acid sequence having at least 60% identity at the amino acid level with SEQ ID NO: 10 and which has an ω3-desaturase activity.

20. The process of claim 18, wherein the nucleic acid sequence which encodes a polypeptide with Δ12-desaturase activity comprises a nucleic acid sequence selected from the group consisting of:
a) a nucleic acid sequence with the sequence shown in SEQ ID NO: 11;
b) a nucleic acid sequence encoding a polypeptide comprising the amino acid sequence shown in SEQ ID NO: 12; and
c) a nucleic acid sequence encoding a polypeptide comprising an amino acid sequence having at least 60% identity at the amino acid level with SEQ ID NO: 12 and which has a Δ12-desaturase activity.

21. The process of claim 16, wherein the nucleic acid is expressed in vegetative tissue.

22. The process of claim 16, wherein the arachidonic acid or eicosapentaenoic acid or arachidonic, acid and eicosapentaenoic acid is present in the useful plants predominantly as an ester in phospholipid- or triacylglyceride-bound form.

23. The process of claim 21, wherein the arachidonic acid or eicosapentaenoic acid or arachidonic acid and eicosapentaenoic acid is predominantly as an ester in phospholipid-bound form, the arachidonic acid or eicosapentaenoic acid being present in the phospholipid esters in an amount of at least 10% by weight based on tie total lipids.

24. The process of claim 21, wherein the arachidonic acid or eicosapentaenoic acid or arachidonic acid and eicosapentaenoic acid is predominantly as an ester in triacylglyceride-bound form, the arachidonic acid or eicosapentaenoic acid being present in the triacylglyceride esters in an amount of at least 10% by weight based on the total lipids.

25. The process of claim 16, wherein a fatty acid selected from the group of the fatty acids consisting of γ-linolenic acid [C18:3^{Δ9, 12, 15}] and dihomo-γ-linolenic acid [C20:3^{Δ6, 9, 12, 15}] is increased by at least 10% in comparison with the nontransgenic wild type plant, in addition to the fatty acids arachidonic acid or eicosapentaenoic acid or arachidonic acid and eicosapentaenoic acid.

26. The process of claim 16, wherein the useful plant is an oil-producing plant, a vegetable plant, a lettuce plant, or an ornamental.

27. The process of claim 16, wherein the arachidonic acid or eicosapentaenoic acid or arachidonic acid and eicosapentaenoic acid are isolated from the useful plants in the form of their oils, lipids, or free fatty acids.

28. The process of claim 16, wherein one or more additional further biosynthesis genes of the fatty acid or lipid metabolism selected from the group acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, allene-oxide synthases, hydroperoxide lyases, and fatty acid elongase(s) is additionally introduced into the useful plants.

29. The process of claim 28, wherein the additional biosynthesis gene of the fatty acid or lipid metabolism is selected from the group Δ4-desaturase, Δ5-desaturase, Δ6-desaturase, Δ8-desaturase, Δ9-desaturase, Δ12-desaturase, Δ6-elongase, and Δ9-elongase.

30. A method for the production of feeding stuffs, foodstuffs, cosmetics or pharmaceuticals comprising utilizing the oils, lipids or free fatty acids produced by the process of claim 27.
